(19) United States
(12) Patent Application Publication
(10) Pub. No.: US 2026/0229319 A1
(43) Pub. Date: Aug. 6, 2026

(54) RECORDING MEDIUM, INFORMATION PROCESSING METHOD, AND INFORMATION PROCESSING DEVICE

(71) Applicant: Fujitsu Limited, Kawasaki-shi (JP)

(72) Inventor: Satoshi IMAMURA, Kawasaki (JP)

(73) Assignee: Fujitsu Limited, Kawasaki-shi (JP)

(21) Appl. No.: 19/455,911

(22) Filed: Jan. 22, 2026

(30) Foreign Application Priority Data
Jan. 31, 2025 (JP) 2025-014774

(51) Int. Cl. G16C 20/50

(52) U.S. Cl. CPC G16C 20/50

(57) ABSTRACT

An information processing device obtains a graph representing a structure of a molecule of interest, the graph including nodes representing respective atom groups of multiple atom groups and edges coupling different nodes. The information processing device refers to a basis function system, calculates, for each edge, an evaluation value representing the magnitude of interaction between atom groups represented by different nodes coupled by the edge, and sets the evaluation value as a weight for the edge. The information processing device divides the graph into portions by cutting edges so that a sum of the weights thereof is minimized and thereby divides the structure of the molecule of interest into fragments corresponding to the portions.

### CROSS REFERENCE TO RELATED APPLICATIONS

This application is based upon and claims the benefit of priority of the prior Japanese Patent Application No. 2025-014774, filed on Jan. 31, 2025, the entire contents of which are incorporated herein by reference.

### FIELD

The embodiments discussed herein are related to a recording medium, an information processing method, and an information processing device.

### BACKGROUND

Conventionally, in the field of drug discovery, material development, or the like, there is a quantum chemical calculation technique for analyzing the structure or properties of a molecule that is a candidate for a drug or a material. In the quantum chemical calculation, for example, the energy of a molecule is calculated. The energy is the ground or excitation energy. Here, in order to reduce the processing amount of the quantum chemical calculation, there is a technique called a density matrix embedding theory in which energy is calculated after dividing a molecular structure into multiple fragments.

In a related art, for example, when the potential energy of a first atom set included in a molecule of interest is calculated, it is specified whether or not to use the atomic weight, the orbital number, and the distance to the center of gravity of the first atom set, for each atom other than the first atom set. For example, refer to Japanese Laid-Open Patent Publication No. 2024-067907.

### SUMMARY

According to an aspect of an embodiment, a computer-readable recording medium stores therein a program for causing a computer to execute a process, the process including: obtaining a graph representing a structure of a molecule of interest, the graph including a plurality of nodes respectively representing a plurality of atom groups of a plurality of atoms forming the molecule of interest, the graph further including a plurality of edges each coupling different nodes of the plurality of nodes; calculating a plurality of evaluation values respectively for the plurality of edges included in the obtained graph, each of the plurality of evaluation values representing a magnitude of an interaction between atom groups represented by the different nodes coupled by the each of the plurality of edges, the each of the plurality of evaluation values being calculated based on atoms belonging to the atom groups represented by the different nodes coupled by the each of the plurality of edges; and dividing the graph into a plurality of portions when the calculated plurality of evaluation values is set as a plurality of weights respectively for each of the plurality of edges included in the obtained graph, the graph being divided into the plurality of portions by cutting any of the plurality of edges such that a sum of respective weights thereof is minimized thereby dividing the structure of the molecule of interest into a plurality of fragments corresponding to the plurality of portions.

The object and advantages of the disclosure will be realized and attained by means of the elements and combinations particularly pointed out in the claims.

It is to be understood that both the foregoing general description and the following detailed description are exemplary and explanatory and are not restrictive of the disclosure.

BRIEF DESCRIPTION OF DRAWINGS

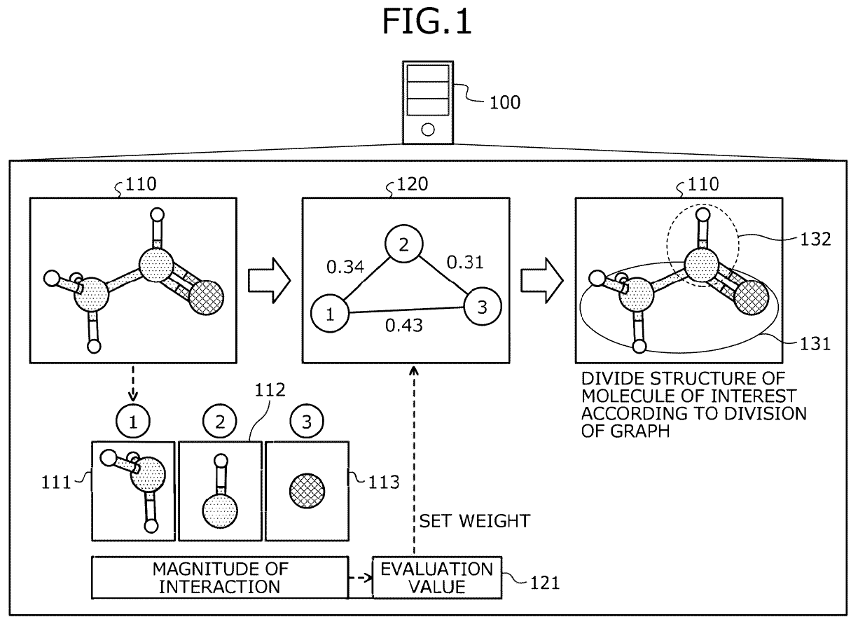

FIG. 1 is an explanatory diagram depicting an example of an information processing method according to an embodiment.

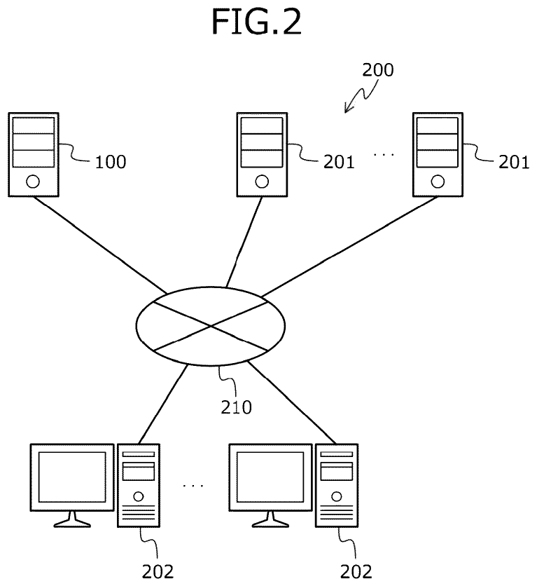

FIG. 2 is an explanatory diagram depicting an example of an information processing system 200.

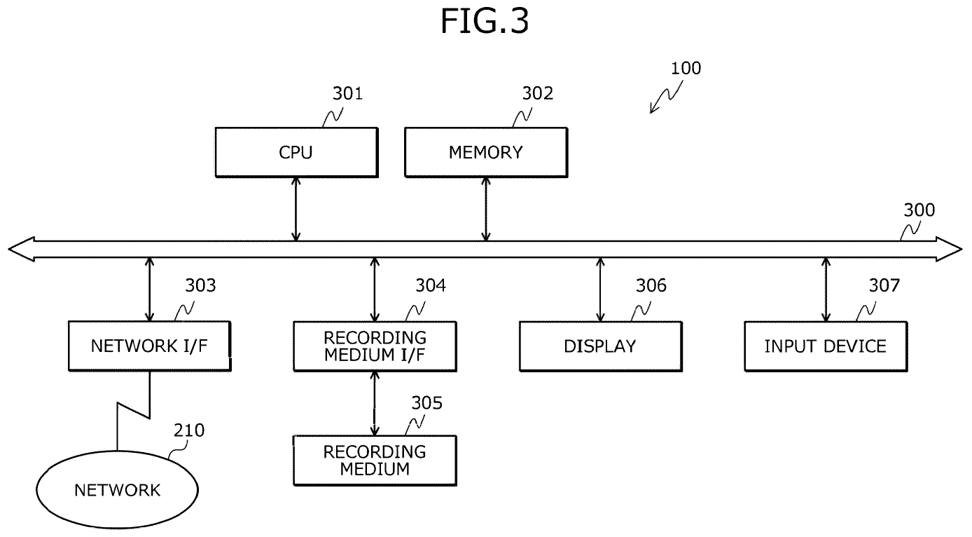

FIG. 3 is a block diagram of an example of a hardware configuration of an information processing device 100.

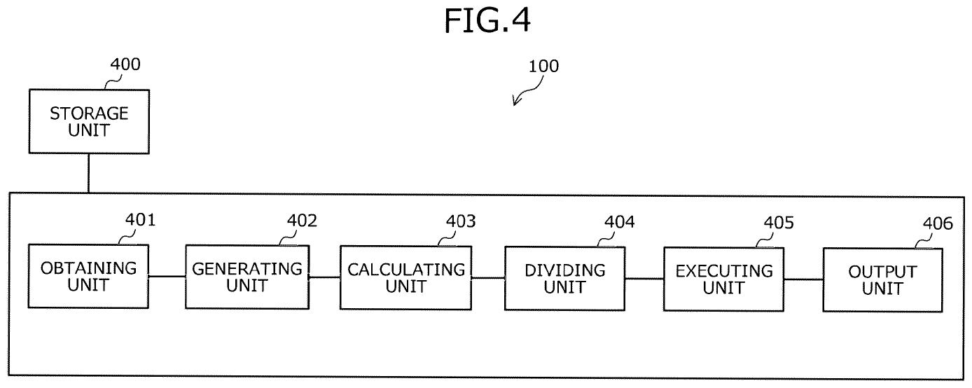

FIG. 4 is a block diagram depicting an example of a functional configuration of the information processing device 100.

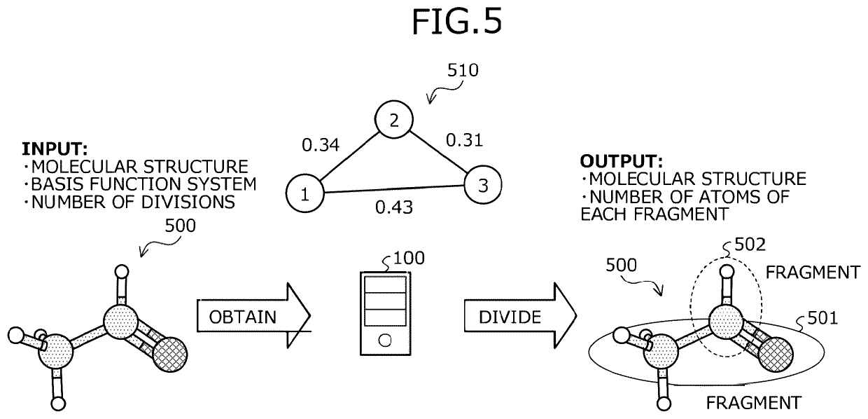

FIG. 5 is an explanatory diagram depicting a flow of operation of the information processing device 100.

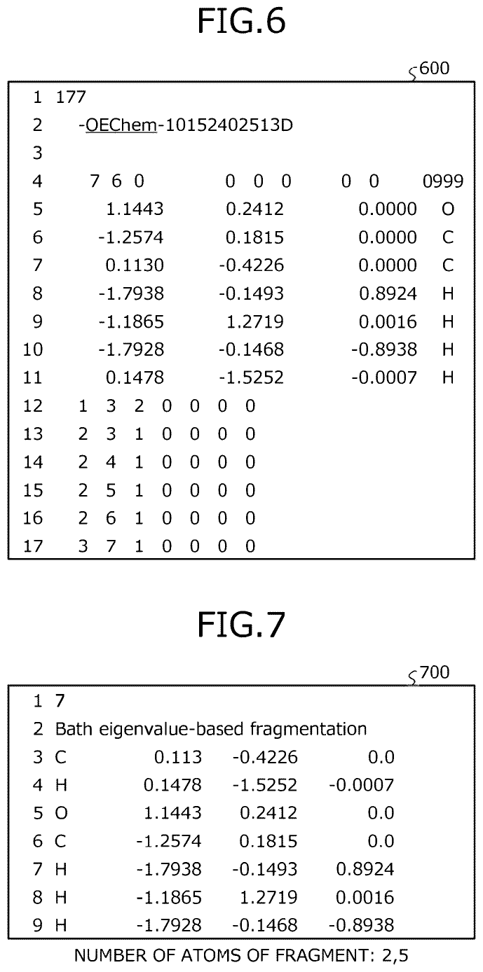

FIG. 6 is an explanatory diagram depicting an example of a first structure data 600.

FIG. 7 is an explanatory diagram depicting an example of a second structure data 700.

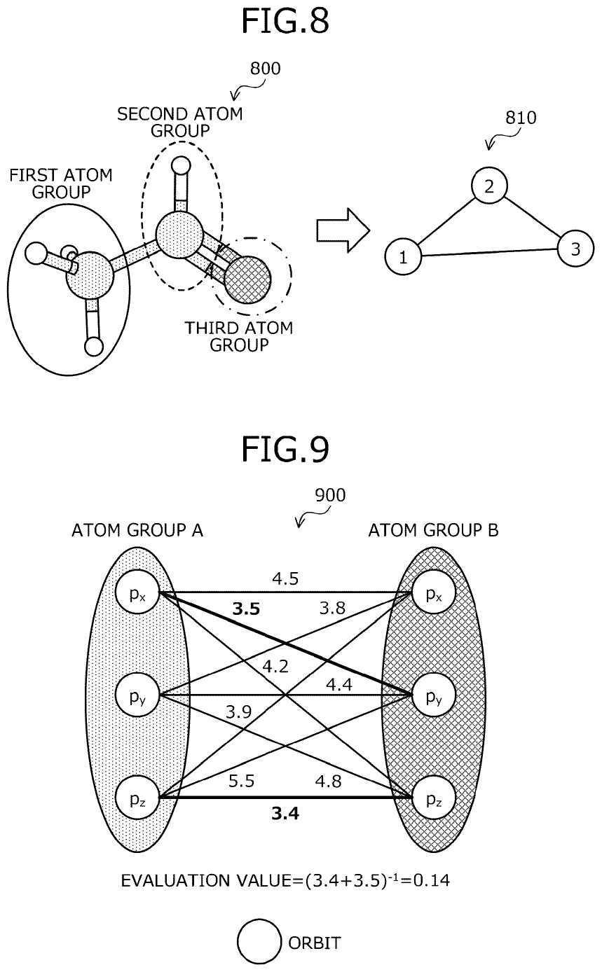

FIG. 8 is an explanatory diagram depicting an example of the operation of the information processing device 100.

FIG. 9 is an explanatory diagram depicting an example of the operation of the information processing device 100.

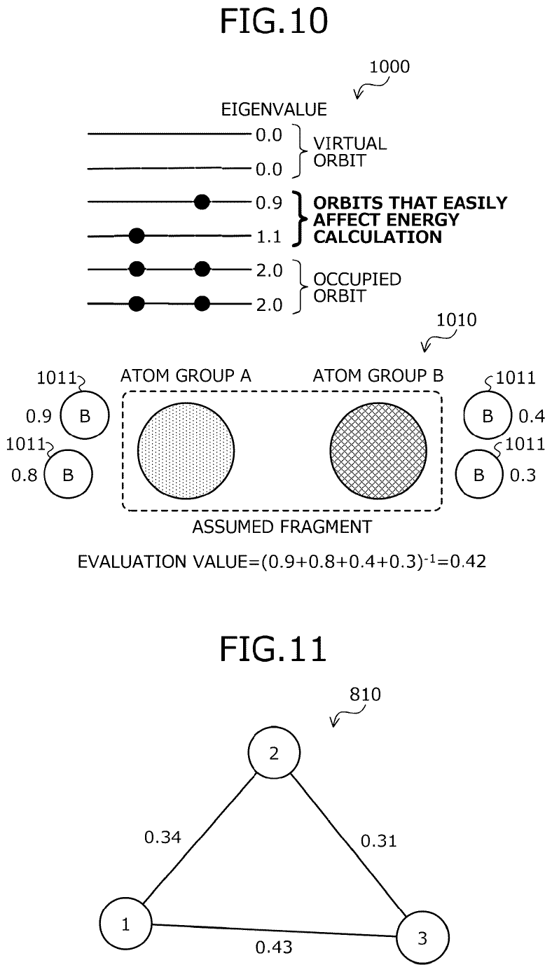

FIG. 10 is an explanatory diagram depicting an example of the operation of the information processing device 100.

FIG. 11 is an explanatory diagram depicting an example of the operation of the information processing device 100.

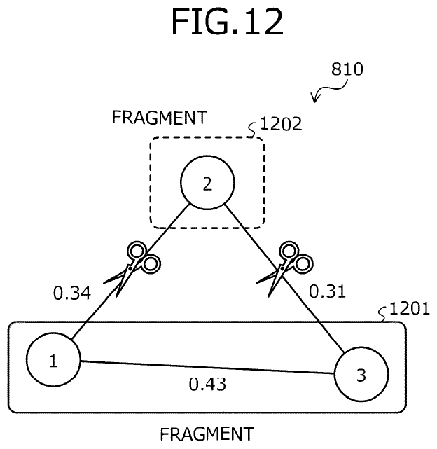

FIG. 12 is an explanatory diagram depicting an example of the operation of the information processing device 100.

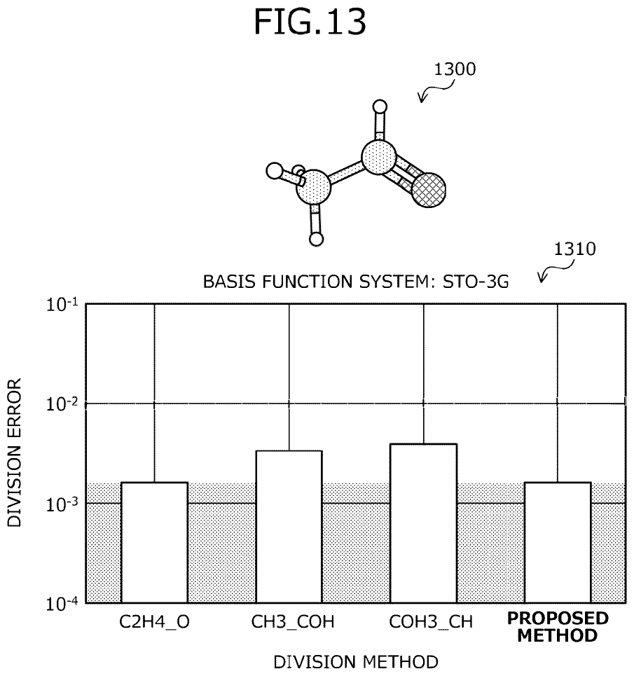

FIG. 13 is an explanatory diagram depicting an example of an effect.

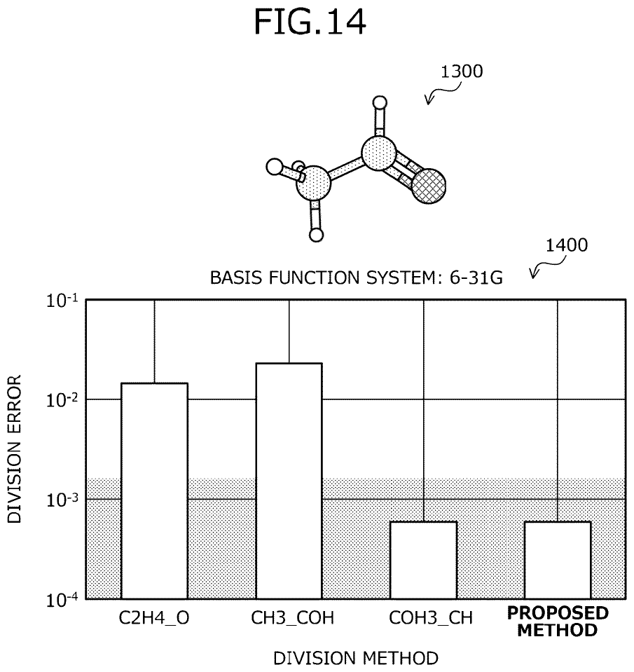

FIG. 14 is an explanatory diagram depicting an example of an effect.

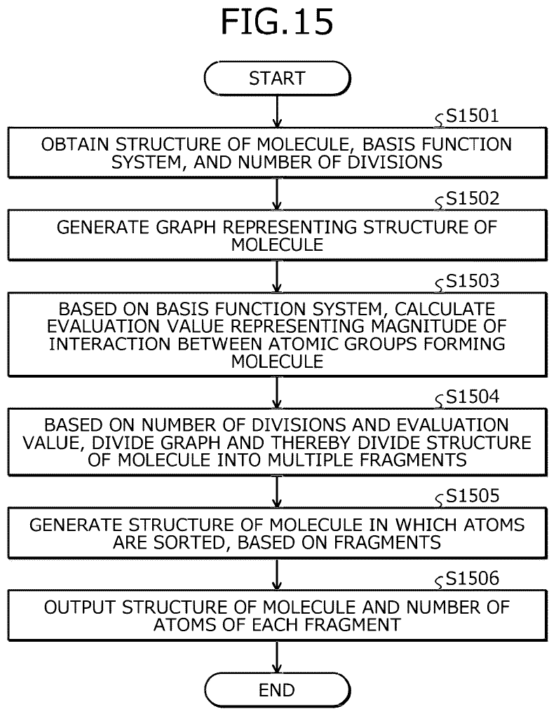

FIG. 15 is a flowchart depicting an example of an overall processing procedure.

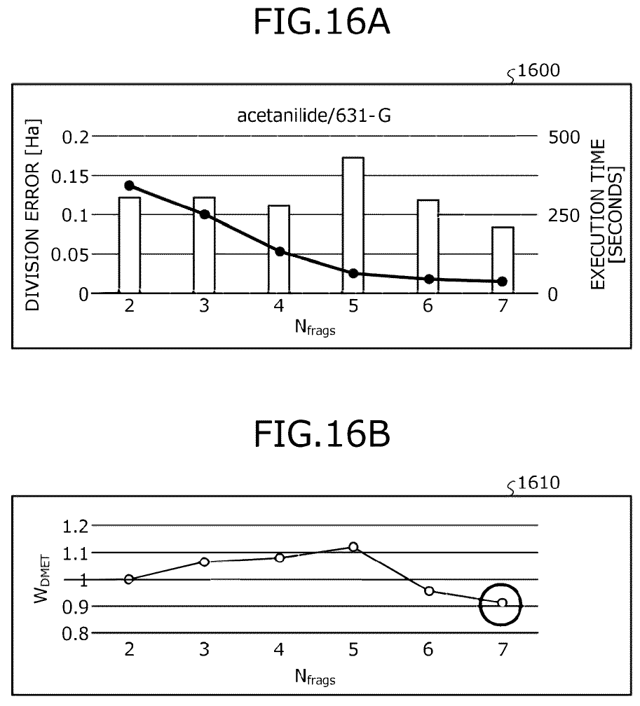

FIG. 16A is an explanatory diagram depicting another example of the operation of the information processing device 100.

FIG. 16B is an explanatory diagram depicting another example of the operation of the information processing device 100.

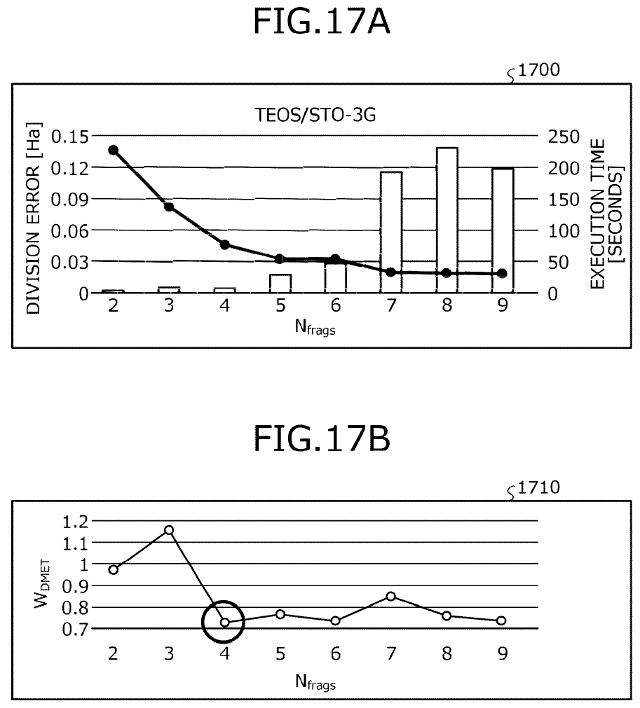

FIG. 17A is an explanatory diagram depicting another example of the operation of the information processing device 100.

FIG. 17B is an explanatory diagram depicting another example of the operation of the information processing device 100.

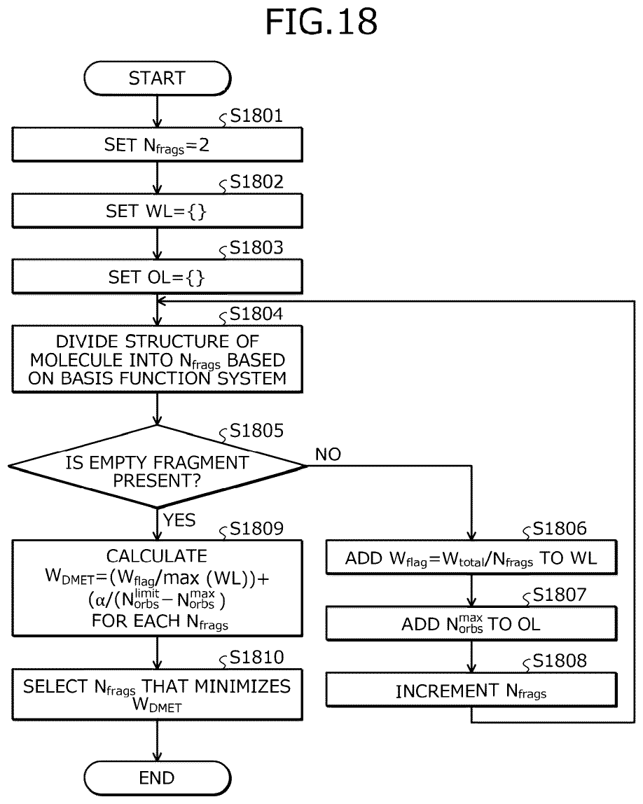

FIG. 18 is a flowchart depicting an example of a procedure of a dividing process.

### DESCRIPTION OF EMBODIMENTS

First, problems associated with the conventional techniques are discussed. In the related art, when the density matrix embedding theory is used, the accuracy of calculating the energy of the molecule may decrease. For example, when the structure of a molecule is not properly divided into multiple fragments, the accuracy of calculating the energy of the molecule is reduced.

Embodiments of a recording medium, an information processing method, and an information processing device according to the present disclosure are described in detail with reference to the accompanying drawings.

FIG. 1 is an explanatory diagram depicting an example of an information processing method according to an embodiment. An information processing device 100 is a computer for appropriately dividing the structure of a molecule into multiple fragments and improving the accuracy of calculating the energy of the molecule when performing quantum chemical calculation using the density matrix embedding theory. The information processing device 100 is, for example, a server or a personal computer (PC).

Conventionally, in the field of drug discovery, material development, or the like, it is desired to perform quantum chemical calculation for calculating the energy of a molecule. The energy is the ground or excitation energy. Here, there is a tendency that the larger the scale of the molecule is, the larger the processing amount necessary for calculating the energy of the molecule is. Specifically, depending on the basis function system, as the scale of the problem increases, the amount of processing necessary to calculate the energy of the molecule tends to increase.

Further, as the accuracy of calculating the energy of the molecule is improved, the amount of processing necessary for calculating the energy of the molecule tends to increase. Specifically, among multiple methods that are approximate solution methods for calculating an approximate solution of the energy of a molecule, a method with relatively high accuracy for calculating an approximate solution tends to need a larger amount of processing for calculating the energy of the molecule than a method with relatively low accuracy for calculating an approximate solution.

Examples of an approximate solution method include a Hartree Fock (HF) method, a Meler-Preset method, and a coupled cluster (CC) method. Specifically, the CC method for calculating the energy of a molecule with high accuracy and a full configuration interaction (FCI) method, which is an exact solution, tend to require a larger amount of processing for calculating the energy of a molecule than other solutions.

For this reason, it is desirable to reduce the amount of processing necessary when performing the quantum chemical calculation. On the other hand, there is a technique called a density matrix embedding theory in which the structure of a molecule is divided into multiple fragments and then the energy of the molecule is calculated in order to reduce the amount of processing necessary when quantum chemical calculation is performed. The density matrix embedding theory is also called density matrix embedding theory (DMET). In the following description, the density matrix embedding theory may be referred to as “DMET”.

Specifically, in the DMET, calculation of the number of electrons of each fragment and the energy of each fragment based on the embedded Hamiltonian is repeated while updating a variational parameter so that the total number of electrons of each of the fragments matches the number of electrons of the entire molecule. The embedded Hamiltonian includes a bath orbit that represents the surrounding environment of each fragment. In the DMET, specifically, the sum of the energies of the fragments is the energy of the entire molecule.

Here, in the DMET, the accuracy of calculating the energy of the entire molecule depends on how the structure of the molecule is divided into multiple fragments. For example, as the number of divisions decreases, the accuracy of calculating the energy of the entire molecule tends to increase. On the other hand, the larger the number of divisions is, the smaller the processing amount necessary for performing the quantum chemical calculation tends to be. In addition, it is considered that the accuracy of calculating the energy of the entire molecule varies depending on the pattern of dividing the structure of the molecule into multiple fragments even when the number of divisions is the same.

Therefore, even when the DMET is used, it may be difficult to maintain the accuracy of calculating the energy of the entire molecule while reducing the processing amount. For example, it is not clear how to divide the structure of a molecule into multiple fragments in order to maintain the accuracy of calculating the energy of the entire molecule while reducing the amount of processing. Specifically, conventionally, a method of dividing the structure of a molecule into multiple fragments so as to maintain the accuracy of calculating the energy of the entire molecule while reducing the processing amount has not been proposed.

Specifically, the larger the scale of the molecule, the larger is the number of patterns for dividing the structure of the molecule into multiple fragments. Depending on the basis function system, how the structure of a molecule is preferably divided into multiple fragments may be different. Therefore, it is difficult to examine along which pattern the molecular structure should be divided into multiple fragments according to the basis function system.

Specifically, there is a problem in that an increase in the work load and work time on the worker is incurred when considering which pattern the molecular structure should be divided into multiple fragments according to the basis function system. In addition, depending on the knowledge of the worker regarding the molecule, the basis function system, the DMET, or the like, the structure of the molecule may not be appropriately divided into multiple fragments.

Therefore, in the present embodiment, an information processing method capable of improving the accuracy of calculating the energy of a molecule using the DMET will be described. Specifically, according to the information processing method, it is possible to appropriately divide the structure of the molecule into multiple fragments according to the basis function system so as to maintain the accuracy of calculating the energy of the entire molecule while reducing the processing amount of the quantum chemical calculation.

In FIG. 1, the information processing device 100 obtains a graph 120 representing a structure 110 of a molecule of interest. A molecule of interest includes multiple atoms. In the example depicted in FIG. 1, it is assumed that the molecule of interest is specifically acetaldehyde. The molecular formula of acetaldehyde is specifically C₂H₄O. For example, each of the multiple atoms is distributed to any atom group of multiple atom groups. An atom group includes, for example, an atom other than hydrogen and a hydrogen atom directly coupled to the atom other than hydrogen. The atom group may contain, for example, only one atom.

In the example depicted in FIG. 1, a structure 110 of a molecule of interest specifically includes three atom groups 111 to 113. Specifically, the atom group 111 includes one C atom and three H atoms directly coupled to the C atom. Specifically, the atom group 112 includes one C atom and one H atom directly coupled to the C atom. The atom group 113 specifically includes an O atom.

The graph 120 includes nodes representing respective atom groups of the multiple atom groups, and edges coupling different nodes. The graph 120 includes, for example, for every pair of nodes, an edge coupling the pair of nodes. For example, the graph 120 may include, for only some pairs of nodes rather than all pairs of nodes, an edge coupling the pair of nodes, and may not include, for any pair of nodes, an edge coupling the pair of nodes. A pair is a set including two different nodes included in a certain graph.

In the example depicted in FIG. 1, the graph 120 specifically includes node 1 corresponding to atom group 111, node 2 corresponding to atom group 112, and node 3 corresponding to atom group 113. Specifically, the graph 120 includes an edge coupling the nodes 1 and 2, an edge coupling the nodes 2 and 3, and an edge coupling the nodes 1 and 3.

For example, the information processing device 100 obtains the graph 120 by generating the graph 120 based on the structure 110 of the molecule of interest. Specifically, the information processing device 100 generates the graph 120 by distributing multiple atoms included in a molecule of interest to multiple atom groups. For example, the information processing device 100 may obtain the graph 120 by receiving an input of the graph 120 based on an operation input of the user. For example, the information processing device 100 may obtain the graph 120 by receiving the graph 120 from another computer.

(1-1) The information processing device 100 refers to a basis function system to calculate, for each edge, an evaluation value 121 representing the magnitude of interaction between atom groups represented by different nodes coupled by the edge, based on atoms belonging to the atom group.

The evaluation value 121 is calculated based on, for example, a Coulomb interaction distance between atoms belonging to different atom groups. The evaluation value 121 may be calculated based on, for example, a bath orbit eigenvalue in a case where different atom groups are fragments. Accordingly, the information processing device 100 may set the weight for the edge with consideration of the basis function system, and may obtain a guideline for dividing the graph 120.

(1-2) The information processing device 100 sets the calculated evaluation value 121 as a weight for each edge included in the obtained graph 120. The information processing device 100 divides the graph 120 into multiple portions so that the total value of the weights for the edges to be cut when dividing the graph 120 is minimized. The number of divisions of the graph 120 is set in advance by the user, for example. The number of divisions of the graph 120 may be dynamically determined, for example. For example, the information processing device 100 uses a solver that solves an integer programming problem to divide the graph 120 into portions corresponding to the set number of divisions so that the total value of weights for edges to be cut is minimized. The solver is, for example, software suitable for solving a minimum cut problem.

In the example depicted in FIG. 1, the number of divisions of the graph 120 is specifically set in advance by the user and is assumed to be 2. Specifically, the information processing device 100 cuts the edge coupling the nodes 1 and 2 and the edge coupling the nodes 2 and 3, and divides the graph 120 into two portions, i.e., a portion including the nodes 1 and 3 and a portion including the node 2. Accordingly, the information processing device 100 may determine how the structure 110 of the molecule of interest is preferably divided via the graph 120.

The information processing device 100 divides the structure 110 of the molecule of interest into multiple fragments corresponding to multiple portions obtained by dividing the graph 120. The multiple fragments correspond to different portions forming the graph 120. In the example depicted in FIG. 1, the information processing device 100 divides the structure 110 of the molecule of interest into a fragment 131 corresponding to a portion including the nodes 1 and 3 and a fragment 132 corresponding to a portion including the node 2. Specifically, the fragment 131 includes one C atom, three H atoms directly coupled to the C atom, and an O atom. The fragment 132 specifically includes one C atom and one H atom directly coupled to the C atom.

Accordingly, the information processing device 100 may appropriately divide the structure 110 of the molecule of interest into multiple fragments, and may identify multiple fragments suitable for quantum chemical calculation using the DMET. Therefore, the information processing device 100 may easily maintain the accuracy of calculating the energy of the entire molecule while reducing the processing amount when performing the quantum chemical calculation using the DMET.

For example, the information processing device 100 may divide the structure 110 of the molecule of interest such that atom groups having a relatively small magnitude of interaction belong to different fragments, and may easily maintain the accuracy of calculating the energy of the molecule. In addition, for example, the information processing device 100 may consider a basis function system when calculating the evaluation value 121. Therefore, for example, the information processing device 100 may appropriately divide the structure 110 of the molecule of interest into multiple fragments according to the basis function system, and may easily maintain the accuracy of calculating the energy of the molecule.

In addition, the information processing device 100 may appropriately divide the structure 110 of the molecule of interest even when the scale of the molecule of interest is large and the number of patterns for dividing the structure 110 of the molecule of interest into multiple fragments is large. Therefore, the information processing device 100 may reduce the workload and the work time on the user who desires to divide the structure 110 of the molecule of interest into a plurality of fragments. In addition, the information processing device 100 may appropriately divide the structure 110 of the molecule of interest into multiple fragments independent of the knowledge of the user regarding the molecule, the basis function system, the DMET, or the like.

Here, while a case in which the functions as the information processing device 100 are realized by a single computer has been described, the present disclosure is not limited hereto. For example, a function of the information processing device 100 may be realized by cooperation among multiple computers. For example, a function of the information processing device 100 may be implemented on a cloud.

Next, an example of an information processing system 200 to which the information processing device 100 depicted in FIG. 1 is applied will be described with reference to FIG. 2.

FIG. 2 is an explanatory diagram depicting an example of the information processing system 200. In FIG. 2, the information processing system 200 includes the information processing device 100, one or more chemical calculation devices 201, and one or more client devices 202.

In the information processing system 200, the information processing device 100 and the one or more chemical calculation device 201 are coupled via a wired or wireless network 210. The network 210 is, for example, a local area network (LAN), a wide area network (WAN), the Internet, or the like. In the information processing system 200, the information processing device 100 and the one or more client devices 202 are coupled via the wired or wireless network 210.

The information processing device 100 is a computer configured to divide the structure of a molecule into multiple fragments. The information processing device 100 obtains a processing request requesting execution of quantum chemical calculation for a molecule of interest using the DMET. Quantum chemical calculations include, for example, calculating the energy of a molecule of interest. The processing request includes, for example, the structure of the molecule of interest.

The structure of the molecule of interest includes, for example, coordinates of each atom of multiple atoms forming the molecule of interest. The structure of the molecule of interest includes, for example, the atom type of each of multiple atoms forming the molecule of interest. The processing request may include, for example, the number of divisions.

The information processing device 100 obtains the structure of the molecule of interest based on the processing request. The information processing device 100 generates a graph representing the structure of the molecule of interest based on the structure of the molecule of interest. The information processing device 100 refers to the basis function system and calculates, for each edge included in the graph, an evaluation value representing the magnitude of interaction between atom groups represented by different nodes coupled by the edge.

The information processing device 100 sets the calculated evaluation value as a weight for each edge included in the graph. The information processing device 100 divides the graph into multiple portions so that the total value of weights for edges to be cut when the graph is divided is minimized. The information processing device 100 divides the structure of the molecule of interest into multiple fragments corresponding to the multiple portions obtained by dividing the graph. Specific examples of the division will be described later with reference to FIGS. 5 to 18.

The information processing device 100 outputs the multiple fragments. The output format is, for example, display on a display, print output to a printer, transmission to another computer, or storage in a storage area. The other computer is, for example, the chemical calculation device 201. The information processing device 100 transmits to any of the chemical calculation devices 201, for example, a calculation request requesting execution of quantum chemical calculation for a molecule of interest using the DMET. The calculation request includes, for example, the multiple fragments. The calculation request includes the structure of the molecule of interest.

The information processing device 100 receives a result of performing quantum chemical calculation for a molecule of interest from any of the chemical calculation devices 201. The information processing device 100 outputs a result of performing quantum chemical calculation for a molecule of interest. The information processing device 100 transmits to the one or more client devices 202, for example, a result of performing quantum chemical calculation for a molecule of interest. For example, the information processing device 100 may output a result of performing quantum chemical calculation for a molecule of interest so that the user may refer to the result. The information processing device 100 is, for example, a server or a PC.

Each of the one or more chemical calculation devices 201 is a computer that performs quantum chemical calculation for molecules. Any of the one or more of the chemical calculation devices 201 receives, from the information processing device 100, a calculation request requesting execution of quantum chemical calculation for a molecule of interest using the DMET. The chemical calculation device 201 obtains the structure of the molecule of interest and multiple fragments, based on the calculation request.

The chemical calculation device 201 performs quantum chemical calculation for the molecule of interest using the DMET, based on the structure of the molecule of interest and the multiple fragments. The chemical calculation device 201 may perform quantum chemical calculation for a molecule of interest by parallel processing using the DMET in cooperation with another one of the chemical calculation devices 201, based on the structure of the molecule of interest and the multiple fragments.

The chemical calculation device 201 generates a result of performing quantum chemical calculation for the molecule of interest. The result includes, for example, the energy of the molecule of interest. The energy is, for example, ground energy or excitation energy. The results may include, for example, the number of electrons in the molecule of interest. For example, the chemical calculation device 201 may communicate with another one of the chemical calculation devices 201 and generate a result of performing quantum chemical calculation for the molecule of interest.

The chemical calculation device 201 transmits the result of performing quantum chemical calculation for the molecule of interest to the information processing device 100. Each of the chemical calculation devices 201 is, for example, a server or a PC. The chemical calculation device 201 may be, for example, a quantum computer.

Each of the client devices 202 is a computer utilized by a user who desires to perform a quantum chemical calculation for a molecule of interest. The user is, for example, a worker. The client device 202 generates a processing request requesting execution of quantum chemical calculation for a molecule of interest using the DMET in response to an operation input of the user. The client device 202 obtains the structure of the molecule of interest, for example, in response to an operation input by the user. The client device 202 generates, for example, a processing request including the structure of the molecule of interest.

The client device 202 transmits the generated processing request to the information processing device 100. The client device 202 receives, from the information processing device 100, a result of performing the quantum chemical calculation for the molecule of interest. The client device 202 outputs the result of performing the quantum chemical calculation for the molecule of interest so that the user may refer to the result. Each of the client devices 202 is, for example, a PC, a tablet terminal, or a smartphone.

Here, while a case in which the information processing device 100 is a device different from the chemical calculation device 201 has been described, the present disclosure is not limited hereto. For example, the information processing device 100 may have a function as the chemical calculation device 201 and may also operate as the chemical calculation device 201. In this case, the information processing system 200 may omit the chemical calculation device 201.

Here, while a case in which the information processing device 100 is a device different from the client device 202 has been described, the present disclosure is not limited hereto. For example, the information processing device 100 may have a function as the client device 202 and may also operate as the client device 202. In this case, the information processing system 200 may omit the client device 202.

Next, with reference to FIG. 3, an example of a hardware configuration of the information processing device 100 is described.

FIG. 3 is a block diagram of an example of the hardware configuration of the information processing device 100. In FIG. 3, the information processing device 100 has a central processing unit (CPU) 301, a memory 302, and a network interface (I/F) 303. The information processing device 100 also has a recording medium I/F 304, a recording medium 305, a display 306, and an input device 307. Further, the components are connected to each other by a bus 300.

Here, the CPU 301 governs overall control of the information processing device 100. The memory 302, for example, includes a read-only memory (ROM), a random-access memory (RAM), and a flash-ROM. In particular, for example, the flash-ROM and/or ROM stores therein various programs and the RAM is used as a work area of the CPU 301. Programs stored to the memory 302 are loaded onto the CPU 301, whereby encoded processes are executed by the CPU 301.

The network I/F 303 is connected to the network 210 via a communications line and is connected to other computers through the network 210. Further, the network I/F 303 administers an internal interface with the network 210 and controls the input and output of data with respect to the other computers. The network I/F 303, for example, is a modem, a LAN adapter, or the like.

The recording medium I/F 304 controls the reading and writing of data with respect to the recording medium 305 under the control of the CPU 301. The recording medium I/F 304 is, for example, a disk drive, a solid-state drive (SSD), a universal serial bus (USB) port, or the like. The recording medium 305 is a nonvolatile memory storing data written thereto under the control of the recording medium I/F 304. The recording medium 305 is, for example, a disk, a semiconductor memory, a USB memory, or the like. The recording medium 305 may be removable from the information processing device 100.

The display 306 displays data such as a cursor, icons, toolboxes, documents, images, or functional information. The display 306 is, for example, a cathode ray tube (CRT), a liquid crystal display, or an organic electroluminescence (EL) display. The input device 307 includes keys for inputting characters, numbers, or various instructions, and inputs data. The input device 307 is, for example, a keyboard or a mouse. The input device 307 may be, for example, a touch panel-type input pad, a numeric keypad, or the like.

The information processing device 100 may include, for example, a camera in addition to the above-described components. Further, the information processing device 100 may include, for example, a printer, a scanner, a microphone, a speaker, or the like in addition to the above-described components. The information processing device 100 may include, for example, the recording medium I/F 304 and/or the recording medium 305 in plural. The information processing device 100 may omit, for example, the display 306 and/or the input device 307. The information processing device 100 may omit the recording medium I/F 304 and the recording medium 305, for example.

An example of a hardware configuration of the chemical calculation devices 201, for example, is a same as the example of the hardware configuration of the information processing device 100 depicted in FIG. 3 and thus, description thereof is omitted herein.

An example of a hardware configuration of the client devices 202, for example, is a same as the example of the hardware configuration of the information processing device 100 depicted in FIG. 3 and thus, description thereof is omitted herein.

Next, an example of a functional configuration of the information processing device 100 will be described with reference to FIG. 4.

FIG. 4 is a block diagram depicting an example of a functional configuration of the information processing device 100. The information processing device 100 includes a storage unit 400, an obtaining unit 401, a generating unit 402, a calculating unit 403, a dividing unit 404, an executing unit 405, and an output unit 406.

The storage unit 400 is realized by, for example, a storage area such as the memory 302 or the recording medium 305 depicted in FIG. 3. Hereinafter, while a case where the storage unit 400 is included in the information processing device 100 will be described, the present disclosure is not limited hereto. For example, the storage unit 400 may be included in a device different from the information processing device 100, and the stored content of the storage unit 400 may be referred to from the information processing device 100.

The obtaining unit 401 to the output unit 406 function as an example of a controller. Specifically, functions of the obtaining unit 401 to the output unit 406 are realized, for example, by causing the CPU 301 to execute a program stored in a storage area such as the memory 302 or the recording medium 305 depicted in FIG. 3 or by the network I/F 303. The processing result of each functional unit is stored in, for example, a storage area such as the memory 302 or the recording medium 305 depicted in FIG. 3.

The storage unit 400 stores various types of information referred to or updated in the processes of the functional units. The storage unit 400 stores, for example, a structure of a molecule of interest. A molecule of interest includes multiple atoms. The structure of the molecule of interest includes, for example, coordinates of each atom of the multiple atoms forming the molecule of interest. The structure of the molecule of interest includes, for example, the atom type of each of the multiple atoms forming the molecule of interest. The structure of the molecule of interest is obtained by, for example, the obtaining unit 401.

The storage unit 400 obtains, for example, a graph representing the structure of the molecule of interest. The graph includes nodes representing respective atom groups of multiple atom groups forming the molecule of interest, and edges coupling different nodes. The graph is, for example, a model including, for every pair of nodes, an edge coupling the pair of nodes.

The graph may be, for example, a model that includes, for only some pairs of nodes rather than all pairs of nodes, edges coupling the nodes forming the pairs. In other words, the graph may be, for example, a model that does not include, for any pair of nodes, an edge coupling the pair of nodes.

An atom group is a group in which at least one atom of the multiple atoms forming the molecule of interest is distributed. The atom group includes, for example, an atom other than hydrogen and a hydrogen atom directly coupled to the atom other than hydrogen. The atom group may contain, for example, only one atom. The edge has, for example, no directivity. The graph is obtained by being generated by the generating unit 402, for example. The graph may be obtained by the obtaining unit 401, for example.

The storage unit 400 stores, for example, a basis function system. The basis function system is a set of functions representing molecular orbitals. The basis function system is, for example, cc-pV5Z, cc-pVQZ, cc-pVTZ, cc-pVDZ, or STO-3G. The basis function system is obtained by, for example, the obtaining unit 401.

The storage unit 400 stores, for example, the number of divisions. The number of divisions indicates, for example, the number of divisions into which the structure of the molecule of interest is divided. The number of divisions is obtained by the obtaining unit 401, for example. The number of divisions may be set in advance by the user, for example.

The obtaining unit 401 obtains various types of information used for the processes of the functional units. The obtaining unit 401 stores the obtained various types of information to the storage unit 400 or outputs the obtained various types of information to the functional units. In addition, the obtaining unit 401 may output various types of information stored in the storage unit 400 to the functional units. The obtaining unit 401 obtains various types of information based on, for example, an operation input of the user. For example, the obtaining unit 401 may receive various types of information from a device different from the information processing device 100.

The obtaining unit 401 obtains, for example, a processing request for dividing the structure of the molecule of interest into multiple fragments. The processing request may further request to perform quantum chemical calculation for the molecule of interest using the DMET. The processing request may include, for example, the structure of the molecule of interest. The processing request may include, for example, a graph representing the structure of the molecule of interest. The processing request may include, for example, a basis function system. The processing request may include, for example, the number of divisions. Specifically, the obtaining unit 401 obtains the processing request by receiving an input of the processing request based on an operation input of the user. Specifically, the obtaining unit 401 may obtain the processing request by receiving the processing request from another computer. The other computer is, for example, the client device 202.

The obtaining unit 401 obtains, for example, the structure of the molecule of interest. Specifically, the obtaining unit 401 obtains the structure of the molecule of interest by extracting the structure of the molecule of interest from the processing request. Specifically, the obtaining unit 401 may obtain the structure of the molecule of interest by receiving an input of the structure of the molecule of interest based on an operation input of the user. Specifically, the obtaining unit 401 may obtain the structure of the molecule of interest by receiving the structure of the molecule of interest from another computer. The other computer is, for example, the client device 202.

The obtaining unit 401 may obtain, for example, a graph representing the structure of the molecule of interest. For example, when the generating unit 402 does not generate the graph representing the structure of the molecule of interest, the obtaining unit 401 obtains the graph representing the structure of the molecule of interest. Specifically, the obtaining unit 401 obtains the graph representing the structure of the molecule of interest by extracting the graph representing the structure of the molecule of interest from the processing request. Specifically, the obtaining unit 401 may obtain the graph representing the structure of the molecule of interest by receiving an input of the graph representing the structure of the molecule of interest, based on an operation input by the user. Specifically, the obtaining unit 401 may obtain the graph representing the structure of the molecule of interest by receiving the graph representing the structure of the molecule of interest from another computer. The other computer is, for example, the client device 202.

The obtaining unit 401 may obtain, for example, a basis function system. For example, when the generating unit 402 does not generate the basis function system, the obtaining unit 401 obtains the basis function system. Specifically, the obtaining unit 401 obtains the basis function system by extracting the basis function system from the processing request. Specifically, the obtaining unit 401 may obtain the basis function system by receiving an input of the basis function system based on an operation input of the user. Specifically, the obtaining unit 401 may obtain the basis function system by receiving the basis function system from another computer. The other computer is, for example, the client device 202.

The obtaining unit 401 obtains, for example, the number of divisions. Specifically, the obtaining unit 401 obtains the number of divisions by extracting the number of divisions from the processing request. Specifically, the obtaining unit 401 may obtain the number of divisions by receiving designation of the number of divisions based on an operation input of the user. Specifically, the obtaining unit 401 may obtain the number of divisions by receiving the number of divisions from another computer. The other computer is, for example, the client device 202.

The obtaining unit 401 may receive a start trigger for starting the process of any functional unit. The start trigger is, for example, a predetermined operation input by the user. The start trigger may be, for example, reception of predetermined information from another computer. The start trigger may be, for example, output of predetermined information by any functional unit. For example, the obtaining unit 401 may regarding obtaining the processing request as a start trigger for starting the processes of the generating unit 402, the calculating unit 403, the dividing unit 404, and the executing unit 405.

The generating unit 402 obtains the graph representing the structure of the molecule of interest by generating the graph representing the structure of the molecule of interest. For example, the generating unit 402 distributes the multiple atoms forming the molecule of interest to multiple atom groups. For example, the generating unit 402 generates a graph including nodes representing the respective atom groups of the multiple atom groups and edges coupling at least some different nodes. Accordingly, the generating unit 402 may obtain a graph serving as a guideline for dividing the structure of the molecule of interest into multiple fragments.

Specifically, the generating unit 402 distributes the multiple atoms into multiple atom groups so that each atom group includes an atom other than hydrogen and a hydrogen atom directly coupled to the atom. Specifically, the generating unit 402 generates a graph including nodes representing the respective atom groups of the multiple atom groups and edges coupling different nodes. Accordingly, the generating unit 402 may obtain a graph serving as a guideline for dividing the structure of the molecule of interest into multiple fragments.

In addition, the generating unit 402 may collectively handle hydrogen atoms that tend to be included in a large amount in the target atoms as atoms other than hydrogen. Therefore, the generating unit 402 may reduce the processing amount when dividing the structure of the molecule of interest into multiple fragments. The generating unit 402 may handle hydrogen atoms together with atoms other than hydrogen, and may prepare a graph so that the structure of the molecule of interest may be appropriately divided into multiple fragments from the viewpoint of performing quantum chemical calculation for the molecule of interest using the DMET.

Specifically, the generating unit 402 distributes the atoms to multiple atom groups such that the atoms are distributed to different atom groups such that each atom group includes only one atom. Specifically, the generating unit 402 generates a graph including nodes representing the respective atom groups of the multiple atom groups and edges coupling different nodes. Accordingly, the generating unit 402 may obtain a graph serving as a guideline for dividing the structure of the molecule of interest into multiple fragments. The generating unit 402 may reduce the amount of processing necessary to distribute the atoms to multiple atom groups.

Specifically, when generating a graph, the generating unit 402 may generate the graph so as to include, for all pairs of nodes, edges coupling the nodes forming the pairs. As a result, the generating unit 402 may comprehensively examine patterns for dividing the structure of the molecule of interest into multiple fragments. Thus, the generating unit 402 facilitate appropriately dividing the structure of the molecule of interest into multiple fragments from the viewpoint of performing quantum chemical calculation for the molecule of interest using the DMET.

Specifically, when generating the graph, the generating unit 402 may generate the graph so as to include an edge coupling nodes forming a pair with respect to only a portion of pairs of nodes instead of all pairs of nodes. As a result, the generating unit 402 may reduce the processing amount when dividing the structure of the molecule of interest into multiple fragments.

The calculating unit 403 calculates, for each edge included in the graph obtained by the obtaining unit 401 or the generating unit 402, an evaluation value representing the magnitude of interaction between atom groups represented by different nodes coupled by the edge. For example, according to the basis function system obtained by the obtaining unit 401, the calculating unit 403 calculates an evaluation value representing the magnitude of interaction between atom groups represented by different nodes coupled by respective edges, based on atoms belonging to the atom groups.

Specifically, the calculating unit 403 calculates an evaluation value for each edge based on a Coulomb interaction distance between atoms belonging to different atom groups among atom groups represented by different nodes coupled by the edge according to the basis function system.

More specifically, the calculating unit 403 calculates, for all pairs of atoms belonging to different atom groups, the Coulomb interaction distance regarding the p orbital between the atoms forming the pair. More specifically, the calculating unit 403 calculates the evaluation value by statistically processing the calculated Coulomb interaction distance for each edge. More specifically, the calculating unit 403 calculates, as the evaluation value, the reciprocal of the sum of the two smaller Coulomb interaction distances among the calculated Coulomb interaction distances for each edge.

Here, while a case in which the calculating unit 403 calculates, for all pairs of atoms belonging to different atom groups, the Coulomb interaction distances related to the p orbitals of the paired atoms has been described, the present disclosure is not limited hereto. For example, the calculating unit 403 may calculate, for all pairs of atoms belonging to different atom groups, the Coulomb interaction distances other than the p-orbitals of the paired atoms.

Accordingly, the calculating unit 403 may set the weight for an edge with consideration of the basis function system, and may obtain a guideline for dividing the graph. The calculating unit 403 may appropriately determine which edge is preferably cut, from the viewpoint of performing the quantum chemical calculation for the molecule of interest using the DMET based on the weight according to the basis function system, and may appropriately divide the graph.

Specifically, the calculating unit 403 calculates an evaluation value for each edge included in the obtained graph according to the basis function system, based on a bath orbit eigenvalue in a case where atom groups represented by different nodes coupled by an edge are fragments. The bath orbit eigenvalue takes a value of [0.0,2.0] when calculated using the limited HF method. In this case, the closer the value of the bath orbit eigenvalue is to 1.0, the more likely the bath orbit eigenvalue is to affect the calculation of the molecular energy.

More specifically, the calculating unit 403 calculates a bath orbit eigenvalue e for each bath orbit of one or more bath orbits in a case where atom groups are fragments. More specifically, the calculating unit 403 calculates min(2−e, e) for each bath orbit. min(2−e, e) represents a smaller one of 2−e and e. More specifically, the calculating unit 403 calculates the reciprocal of the total value of the calculated min(2−e, e) as the evaluation value.

Accordingly, the calculating unit 403 may set the weight for an edge with consideration of the basis function system, and may obtain a guideline for dividing the graph. The calculating unit 403 may appropriately determine which edge is preferably cut, from the viewpoint of performing the quantum chemical calculation for the molecule of interest using the DMET, based on the weight according to the basis function system, and may appropriately divide the graph.

The dividing unit 404 divides the graph obtained by the obtaining unit 401 or the generating unit 402 into multiple portions, thereby dividing the structure of the molecule of interest into multiple fragments corresponding to multiple portions. The multiple fragments correspond to different portions. For example, the dividing unit 404 divides the structure of the molecule of interest into a number of fragments corresponding to the number of divisions by dividing the graph into a number of portions corresponding to the number of divisions. The number of divisions is obtained by the obtaining unit 401, for example. The number of divisions is set in advance by the user, for example.

Specifically, when evaluation values calculated by the calculating unit 403 are set respectively as the weights for edges included in the graph, the dividing unit 404 divides the graph into portions corresponding to the number of divisions so that the total value of the weights for the edges to be cut when the graph is divided is minimized. More specifically, the dividing unit 404 divides the graph into the same number of portions as the number of divisions by using a solver that solves an integer programming problem so that the total value of weights for edges to be cut is minimized. The solver is, for example, software suitable for solving a minimum cut problem.

Accordingly, the dividing unit 404 may determine how the structure of the molecule of interest is preferably divided through the graph. The dividing unit 404 may appropriately divide the structure of the molecule of interest into multiple fragments, and may identify multiple fragments suitable for quantum chemical calculation using the DMET. When performing the quantum chemical calculation using the DMET, the dividing unit 404 may easily maintain the accuracy of calculating the energy of the entire molecule while reducing the processing amount.

For example, the dividing unit 404 divides the structure of the molecule of interest into the number of fragments corresponding to a number of candidates by dividing the graph into each number of portions corresponding to each the number of candidates into which the structure of the molecule of interest is to be divided. The number of candidates is the number of divisions. The multiple numbers of candidates are set in advance by the user, for example. As a result, the dividing unit 404 may divide the structure of the molecule of interest into as many fragments as the number of candidates for each of the multiple numbers of candidates.

Specifically, the dividing unit 404 may dynamically change the number of candidates. Specifically, the dividing unit 404 sets the initial value of the number of candidates to 2. Specifically, the dividing unit 404 repeatedly performs a series of processes including dividing the graph into portions corresponding to the current number of candidates until the graph cannot be divided into portions corresponding to the current number of candidates, dividing the structure of the molecule of interest into fragments corresponding to the current number of candidates, and then adding 1 to the number of candidates. As a result, the dividing unit 404 may divide the structure of the molecule of interest into as many fragments as the number of candidates for each of the multiple numbers of candidates.

For example, the dividing unit 404 calculates the degree of contribution to the quantum chemical calculation using the DMET for each candidate number, based on the division result. The contribution degree relates to, for example, at least one of the processing amount of the quantum chemical calculation and the accuracy of the quantum chemical calculation. For example, the dividing unit 404 identifies any number of candidates determined to have the highest calculated degree of contribution among the multiple numbers of candidates. The dividing unit 404 selects, for the identified number of candidates, for example, as many fragments as the identified number of candidates obtained by dividing the structure of the molecule of interest.

As described, the dividing unit 404 may identify any candidate number that is an appropriate number of divisions among the multiple candidate numbers. The dividing unit 404 may divide the structure of the molecule of interest into multiple fragments by an appropriate number of divisions, and may identify multiple fragments suitable for quantum chemical calculation using the DMET.

The executing unit 405 executes a quantum chemical calculation using the DMET, based on the multiple fragments divided by the dividing unit 404. The executing unit 405 generates a result of executing the quantum chemical calculation. The results include, for example, the energy of the molecule of interest. The energy is, for example, ground energy or excitation energy. The results may include, for example, the number of electrons in the molecule of interest. Accordingly, the performing unit 405 may accurately and efficiently perform the quantum chemical calculation.

The executing unit 405 may control another computer to execute quantum chemical calculation using the DMET, based on the multiple fragments divided by the dividing unit 404. The other computer is, for example, one or more of the chemical calculation devices 201. The executing unit 405 receives a result of executing the quantum chemical calculation from the other computer. Accordingly, the performing unit 405 may accurately and efficiently perform the quantum chemical calculation.

The output unit 406 outputs a processing result of at least one of the functional units. The output format is, for example, display on a display, print output to a printer, transmission to an external device by the network I/F 303, or storage in a storage area such as the memory 302 or the recording medium 305. Accordingly, the output unit 406 may notify the user of the processing result of at least one of the functional units, and the convenience of the information processing device 100 may be improved.

The output unit 406 outputs, for example, the multiple fragments divided or selected by the dividing unit 404. Specifically, the dividing unit 404 outputs, in association with information indicating each of the multiple fragments divided or selected by the dividing unit 404, information indicating each of one or more atoms belonging to the fragment. More specifically, the output unit 406 outputs, in association with information indicating each of the multiple fragments, information indicating each of the one or more atoms distributed to the fragment so that the user may refer to the information.

More specifically, the output unit 406 may transmit in association with information indicating each fragment of the multiple fragments, information indicating each atom of the one or more atoms distributed to the fragment, the information being transmitted to another computer. The other computer is, for example, one of the chemical calculation devices 201 or one of the client devices 202. Accordingly, the output unit 406 may make the multiple fragments available externally. Therefore, the output unit 406 may maintain externally the accuracy of the quantum chemical calculation while reducing the processing amount of the quantum chemical calculation.

The output unit 406 outputs, for example, a result of the quantum chemical calculation performed by the performing unit 405. Specifically, the output unit 406 outputs the result of the quantum chemical calculation so that the user may refer to the result. Specifically, the output unit 406 may transmit a result of performing the quantum chemical calculation to another computer. The other computer is, for example, one of the client devices 202. Accordingly, the information processing device 100 may use the result of performing the quantum chemical calculation.

Here, while a case in which the information processing device 100 includes the obtaining unit 401, the generating unit 402, the calculating unit 403, the dividing unit 404, the executing unit 405, and the output unit 406 has been described, the present disclosure is not limited hereto. For example, the information processing device 100 may omit any of the functional units. Specifically, the information processing device 100 may omit the executing unit 405. In this case, specifically, the information processing device 100 may cooperate with another computer operating as the executing unit 405. The other computer is, for example, one of the chemical calculation devices 201.

Next, a flow of operation of the information processing device 100 will be described with reference to FIGS. 5 to 7.

FIG. 5 is an explanatory diagram depicting a flow of operation of the information processing device 100. In FIG. 5, the information processing device 100 obtains input data. The input data includes, for example, a structure 500 of a molecule of interest. Specifically, the input data includes first structure data 600 representing the structure 500 of the molecule of interest. The first structure data 600 includes, for example, a type of each of multiple atoms forming the molecule of interest, coordinates of each atom, and information related to a chemical bond between two atoms. The input data includes, for example, a basis function system. The input data includes, for example, the number of divisions. In the example depicted in FIG. 5, the number of divisions is two. Here, an example of the first structure data 600 will be described with reference to FIG. 6.

FIG. 6 is an explanatory diagram depicting an example of the first structure data 600. In FIG. 6, the first structure data 600 is in a structure data file (SDF) format. The left end of the first structure data 600 is a line number. The first to third lines of the first structure data 600 indicate header information.

The fourth line of the first structure data 600 is a count line. The count line includes, for example, the number of atoms, the number of bonds, a setting value 1, a setting value 2, a setting value 3, a setting value 4, a setting value 5, a setting value 6, and a setting value 7 from the top. In the example depicted in FIG. 6, the setting value 1, the setting value 2, the setting value 3, the setting value 4, the setting value 5, and the setting value 6 are default values 0. The setting value 7 is the default value 0999. Here, the setting value 1, the setting value 2, the setting value 3, the setting value 4, the setting value 5, the setting value 6, and the setting value 7 do not have a specific meaning.

The fifth to (N+4)-th lines of the first structure data 600 indicate the types of atoms and the coordinates of the atoms. In the example depicted in FIG. 6, the types of atoms and the coordinates of the atoms are arranged in descending order of the number of orbitals. N is the number of atoms. The type is, for example, O, C, or H. The coordinates are a combination of an x coordinate value, a y coordinate value, and a z coordinate value in a three-dimensional space.

The (N+5)-th and subsequent lines of the first structure data 600 each indicate a coupled pair and a bond order. A coupled pair indicates atom numbers of the atoms that are paired. In the example depicted in FIG. 6, the 12th line of the first structure data 600 specifically represents a bond between an O atom, which is the first atom, and a C atom, which is the third atom, and specifies an O atom number “1”, a C atom number “3”, and a bond order “2”.

Here, the description continues with reference to FIG. 5. (5-1) The information processing device 100 generates a graph 510 representing the structure 500 of the molecule of interest. For example, based on the first structure data 600, the information processing device 100 distributes the multiple atoms forming the molecule of interest to multiple atom groups so that one atom other than an H atom and an H atom directly bonded to the one atom belong to the same atom group.

In the example depicted in FIG. 5, specifically, the information processing device 100 distributes the first C atom and three H atoms directly bonded to the C atom to a first atom group. Specifically, the information processing device 100 distributes a second C atom and one H atom directly bonded to the C atom to the second atom group. Specifically, the information processing device 100 distributes the O atoms to a third atom group.

For example, the information processing device 100 generates a graph 510 including nodes representing the atom groups respectively and edges coupling the nodes. The graph 510 is, for example, a model that is a complete graph including, for all pairs of nodes, edges coupling the nodes forming the pairs. The graph 510 may be, for example, a model including, for only some pairs of nodes, edges coupling the nodes forming the pairs.

Specifically, information processing device 100 sets node 1 corresponding to the first atom group, node 2 corresponding to the second atom group, and node 3 corresponding to the third atom group. Specifically, the information processing device 100 generates the graph 510 including the set nodes 1 to 3, an edge coupling the nodes 1 and 2, an edge coupling the nodes 2 and 3, and an edge coupling the nodes 1 and 3. Accordingly, the information processing device 100 may divide the structure 500 of the molecule of interest via the graph 510.

(5-2) The information processing device 100 calculates, for each edge included in the graph 510, an evaluation value representing the magnitude of interaction between atom groups represented by different nodes coupled by the edge. For example, the information processing device 100 calculates, for each edge, an evaluation value, based on a Coulomb interaction distance between atoms belonging to different atom groups among atom groups represented by different nodes coupled by the edge, according to the basis function system. For example, the information processing device 100 may calculate, for each edge, an evaluation value based on a bath orbit eigenvalue in a case where atom groups represented by different nodes coupled by the edge are fragments, according to the basis function system.

The information processing device 100 respectively sets the calculated evaluation values as weights for the edges included in the graph 510. Accordingly, the information processing device 100 may set weights for the edges according to the basis function system, and may obtain a guideline for dividing the graph 510. The information processing device 100 may appropriately determine which edge is preferably cut, from the viewpoint of performing the quantum chemical calculation for the molecule of interest using the DMET according to the basis function system, and may appropriately divide the graph 510.

(5-3) The information processing device 100 divides the graph 510 into portions corresponding to the number of divisions, thereby dividing the structure 500 of the molecule of interest into fragments corresponding to the number of divisions. Here, from the viewpoint of maintaining the accuracy of the quantum chemical calculation, it is preferable that atom groups having a relatively large magnitude of interaction belong to the same fragment. Similarly, from the viewpoint of maintaining the accuracy of the quantum chemical calculation, it is preferable that atom groups having a relatively small magnitude of interaction belong to different fragments.

Therefore, when the graph 510 is divided in order to divide the structure 500 of the molecule of interest, it is preferable to cut an edge having a relatively small weight. Therefore, the information processing device 100 divides the graph 510 into portions corresponding to the number of divisions so that the total value of the weights for the edges cut when the graph 510 is divided is minimized. In the example depicted in FIG. 5, specifically, the information processing device 100 cuts an edge coupling the nodes 1 and 2 and an edge coupling the nodes 2 and 3, and divides the graph 510 into two portions, that is, a portion including the nodes 1 and 3 and a portion including the node 2. Accordingly, the information processing device 100 may determine how the structure 500 of the molecule of interest is preferably divided via the graph 510.

In the example depicted in FIG. 5, specifically, according to the result of dividing the graph 510, the information processing device 100 divides the structure 500 of the molecule of interest into a fragment 501 corresponding to a portion including the nodes 1 and 3 and a fragment 502 corresponding to a portion including the node 2. Specifically, the fragment 501 includes one C atom, three H atoms directly coupled to the C atom, and an O atom. Specifically, the fragment 502 includes one C atom and one H atom directly coupled to the C atom. Accordingly, the information processing device 100 may appropriately divide the structure 500 of the molecule of interest into multiple fragments according to the basis function system, and may maintain the accuracy of the quantum chemical calculation while reducing the processing amount of the quantum chemical calculation.

(5-4) The information processing device 100 generates and outputs output data indicating the fragments obtained by dividing the structure 500 of the molecule of interest. Specifically, the output data includes second structure data 700 representing the structure 500 of the molecule of interest. The second structure data 700 includes, for example, the number of atoms of each fragment so that the fragments may be identified. The second structure data 700 indicates, for example, the type of each atom among the atoms forming the molecule of interest and the coordinates of each atom in sequence so that the fragments may be identified. Here, an example of the second structure data 700 will be described with reference to FIG. 7.

FIG. 7 is an explanatory diagram depicting an example of the second structure data 700. In FIG. 7, the left end of the second structure data 700 is a line number. The first line of the second structure data 700 indicates the number of atoms. The second line of the second structure data 700 is a comment column.

The third and subsequent lines of the second structure data 700 indicate the types of atoms and the coordinates of the atoms. In the example depicted in FIG. 7, the types of atoms and the coordinates of the atoms are arranged for each fragment. The type is, for example, O, C, or H. The coordinates are a combination of an x coordinate value, a y coordinate value, and a z coordinate value in a three-dimensional space. The second structure data 700 includes the number of atoms of each fragment. In the example depicted in FIG. 7, the number of atoms of the respective fragments is 2 and 5. Therefore, in the third and subsequent lines of the second structure data 700, two atoms corresponding to the first two lines belong to the same fragment. In the third and subsequent lines of the second structure data 700, five atoms corresponding to the remaining five lines belong to the same fragment.

Accordingly, the information processing device 100 may perform the quantum chemical calculation, based on the output data. For example, the information processing device 100 may appropriately divide the structure 500 of the molecule of interest into multiple fragments according to the basis function system, and may accurately and efficiently perform quantum chemical calculation for the molecule of interest using the DMET. The information processing device 100 may obtain a result of performing the quantum chemical calculation.

Next, an example of the operation of the information processing device 100 will be described with reference to FIGS. 8 to 12.

FIGS. 8, 9, 10, 11, and 12 are explanatory diagrams depicting an example of the operation of the information processing device 100. In FIG. 8, the information processing device 100 obtains input data including the first structure data 600 related to a molecule of interest. In the example depicted in FIG. 8, the molecule of interest is acetaldehyde. The information processing device 100 obtains, based on the first structure data 600, the coordinates and type of each of the atoms forming the molecule of interest and the bonding relationship between the atoms.

(8-1) The information processing device 100 generates a graph 810 representing a structure 800 of the molecule of interest, based on the coordinates and the type of each atom and the bonding relationship between the atoms. For example, the information processing device 100 distributes the atoms forming the molecule of interest to multiple atom groups so that one atom other than an H atom and an H atom directly bonded to the atom belong to the same atom group. In the example depicted in FIG. 8, specifically, the information processing device 100 distributes the first C atom and three H atoms directly bonded to the C atom to the first atom group. Specifically, the information processing device 100 distributes the second C atom and one H atom directly bonded to the C atom to the second atom group. Specifically, the information processing device 100 distributes an O atom to the third atom group.

The information processing device 100 generates, for example, the graph 810 including nodes respectively representing the atom groups and, for all pairs of nodes, edges coupling the nodes forming the pairs. In the example depicted in FIG. 8, the information processing device 100 specifically sets a node 1 corresponding to the first atom group, a node 2 corresponding to the second atom group, and a node 3 corresponding to the third atom group. Specifically, the information processing device 100 generates the graph 810 including the set nodes 1 to 3, an edge coupling the nodes 1 and 2, an edge coupling the nodes 2 and 3, and an edge coupling the nodes 1 and 3. Accordingly, the information processing device 100 may divide the structure 800 of the molecule of interest via the graph 810.

(8-2) The information processing device 100 calculates, for each edge included in the graph 810, an evaluation value representing the magnitude of interaction between atom groups represented by the different nodes coupled by the edge. For example, a larger evaluation value indicates a larger interaction. The evaluation value is calculated based on, for example, a Coulomb interaction distance between atoms belonging to different atom groups or a bath orbit eigenvalue in a case where atom groups are fragments.

First, a case where the information processing device 100 calculates an evaluation value for each edge based on the Coulomb interaction distance between the atoms belonging to different atom groups will be described with reference to FIG. 9.

In FIG. 9, the information processing device 100 calculates, for each edge, according to the basis function system, an evaluation value based on the Coulomb interaction distance d_{pq} between the atoms belonging to different atom groups among atom groups represented by different nodes coupled by the edge. Here, the Coulomb interaction distance d_{pq}=[J_{pq}/(JₚₚJ_{qq})^{1/2}]⁻¹−1. Here, J_{pq} is the Coulomb interaction between the orbit p and the orbit q. Regarding the Coulomb interaction distance, for example, Ye, Hong-Zhou, et al, “Bootstrap embedding for molecules,” Journal of Chemical Theory And Computation, 15.8 (2019): 4497-4506 may be referred to.

For example, for each edge, the information processing device 100 calculates the Coulomb interaction distance d_{pq} related to the p-orbital between atoms for all pairs of atoms belonging to different atom groups among atom groups represented by different nodes coupled by the edge. Since the p-orbitals contribute to double or triple bonds, the p-orbitals are used as a guide for evaluating interactions.

For example, the information processing device 100 calculates the evaluation value by statistically processing the calculated Coulomb interaction distance d_{pq} for each edge. Specifically, the information processing device 100 calculates, as the evaluation value, the reciprocal of the sum of the two smaller Coulomb interaction distances d_{pq} among the calculated Coulomb interaction distances d_{pq} for each edge.

In the example depicted in FIG. 9, it is assumed that the information processing device 100 specifically calculates an evaluation value representing the magnitude of interaction in a pair 900 of an atom group A and an atom group B. The atom group A includes an orbit pₓ, an orbit p_{y}, and an orbit p_{z}. The atom group B includes an orbit pₓ, an orbit p_{y}, and an orbit p_{z}. Specifically, the information processing device 100 calculates the evaluation value=(3.4+3.5)⁻¹=0.14. Accordingly, the information processing device 100 may set the weight for the edge with consideration of the basis function system, and may obtain a guideline for dividing the graph 810.

Next, a case where the information processing device 100 calculates an evaluation value for each edge, based on a bath orbit eigenvalue when atom groups are fragments will be described with reference to FIG. 10.

In FIG. 10, the information processing device 100 calculates, for each edge, an evaluation value based on a bath orbit eigenvalue in a case where atom groups represented by different nodes coupled by the edge are fragments, according to the basis function system. The bath orbit eigenvalue takes a value of [0.0, 2.0] when calculated using the limited HF method.

The closer the value of the bath orbit eigenvalue is to 1.0, the more likely it is to affect the calculation of molecular energy. As indicated by reference numeral 1000, the eigenvalue 0.0 corresponds to the virtual orbit. As indicated by reference numeral 1000, the eigenvalue 2.0 corresponds to the occupied orbit. Further, as indicated by reference numeral 1000, the eigenvalue 0.9 or the eigenvalue 1.1 is an orbit that is likely to affect the calculation of the energy of the molecule.

More specifically, the calculating unit 403 calculates a bath orbit eigenvalue e for each bath orbit of one or more bath orbits in a case where atom groups are fragments. More specifically, the calculating unit 403 calculates min(2−e, e) for each bath orbit. min(2−e, e) represents the smaller one of 2−e and e. More specifically, the calculating unit 403 calculates the reciprocal of the total value of the calculated min(2−e, e) as the evaluation value.

In the example depicted in FIG. 10, it is assumed that the information processing device 100 specifically calculates an evaluation value representing the magnitude of interaction in a pair 1010 of the atom group A and the atom group B. When the pair 1010 of the atom group A and the atom group B is a fragment, there are multiple bath orbits 1011 for considering other fragments.

Specifically, the information processing device 100 calculates min(2−e, e) of each of the bath orbits 1011. Specifically, the information processing device 100 calculates the evaluation value=(0.9+0.8+0.4+0.3)⁻¹=0.42. Accordingly, the information processing device 100 may set the weight for the edge with consideration of the basis function system, and may obtain a guideline for dividing the graph 810. Next, FIG. 11 will be described.

In FIG. 11, the information processing device 100 sets the evaluation values calculated in FIG. 9 or 10 as the weights respectively for the edges included in the graph 810. Accordingly, the information processing device 100 may set the weights for the edges according to the basis function system, and may obtain a guideline for dividing the graph 810. The information processing device 100 may appropriately determine which edge is preferably cut, from the viewpoint of maintaining the accuracy of the quantum chemical calculation according to the basis function system, and may appropriately divide the graph 810.

For example, from the viewpoint of maintaining the accuracy of the quantum chemical calculation, it is preferable that atom groups having a relatively large magnitude of interaction belong to the same fragment. Similarly, from the viewpoint of maintaining the accuracy of the quantum chemical calculation, it is preferable that atom groups having a relatively small magnitude of interaction belong to different fragments.

Therefore, when the graph 810 is divided to divide the structure 800 of the molecule of interest, it is considered that there is a property that an edge having a relatively small weight is preferably cut. Specifically, the information processing device 100 may appropriately determine which edge is preferably cut with consideration of the property, based on the weight for the edge. Next, FIG. 12 will be described.

In FIG. 12, the information processing device 100 divides the graph 810 into portions corresponding to the number of divisions so that the total value Wₜₒₜₐₗ of the weights for the edges cut when the graph 810 is divided is minimized. Here, the number of divisions is 2. The information processing device 100 divides the graph 810 into two parts so that Wₜₒₜₐₗ is minimized, for example, using a function called part_graph of a library called METIS.

In the example depicted in FIG. 12, specifically, the information processing device 100 cuts an edge coupling the nodes 1 and 2 and an edge coupling the nodes 2 and 3, and divides the graph 810 into two portions, that is, a portion including the nodes 1 and 3 and a portion including the node 2. Accordingly, the information processing device 100 may determine how the structure 800 of the molecule of interest is preferably divided via the graph 810.

According to the result of dividing the graph 510, the information processing device 100 divides the molecule of interest structure 500 into a fragment 1201 corresponding to a portion including the nodes 1 and 3 and a fragment 1202 corresponding to a portion including the node 2. Specifically, the fragment 1201 includes one C atom, three H atoms directly coupled to the one C atom, and an O atom. The fragment 1202 specifically includes one C atom and one H atom directly coupled to the one C atom. Accordingly, the information processing device 100 may appropriately divide the structure 800 of the molecule of interest into multiple fragments according to the basis function system, and may maintain the accuracy of the quantum chemical calculation while reducing the processing amount of the quantum chemical calculation. Next, an example of an effect of the information processing device 100 will be described with reference to FIGS. 13 and 14.

FIGS. 13 and 14 are explanatory diagrams depicting examples of an effect. In the examples depicted in FIGS. 13 and 14, it is assumed that the number of divisions is 2. A molecule of interest 1300 is assumed to be acetaldehyde. In the example depicted in FIG. 13, the basis function system is STO-3G. In the example depicted in FIG. 14, it is assumed that the basis function system is 6-31G. First, FIG. 13 will be described.

A graph 1310 in FIG. 13 depicts a division error corresponding to the accuracy of the quantum chemical calculation in the proposed method of dividing the structure of the molecule of interest 1300 by the information processing device 100 in a case where the basis function system is STO-3G. In addition, the graph 1310 depicts a division error corresponding to the accuracy of the quantum chemical calculation when the structure of the molecule of interest 1300 is divided by a specific division pattern with respect to the case where the basis function system is STO-3G.

C2H4_O corresponds to a splitting pattern that splits the structure of the molecule of interest 1300 into two fragments, a fragment containing two C atoms and four H atoms and a fragment containing one O atom. CH3_COH corresponds to a splitting pattern that splits the structure of the molecule of interest 1300 into two fragments, a fragment containing one C atom and three H atoms and a fragment containing one C atom, one O atom and one H atom. COH3_CH corresponds to a splitting pattern that splits the structure of the molecule of interest 1300 into two fragments, a fragment containing one C atom, one O atom, and three H atoms, and a fragment containing one C atom and one H atom. Next, FIG. 14 will be described.

A graph 1400 in FIG. 14 depicts a division error corresponding to the accuracy of the quantum chemical calculation in the proposed method of dividing the structure of the molecule of interest 1300 by the information processing device 100 in the case where the basis function system is 6-31G. In addition, the graph 1400 depicts a division error corresponding to the accuracy of the quantum chemical calculation when the structure of the molecule of interest 1300 is divided by a specific division pattern with respect to the case where the basis function system is 6-31G.

As depicted in the graphs 1310 and 1400, the information processing device 100 may change the division pattern for dividing the structure of the molecule of interest 1300 according to the basis function system for the same molecule of interest 1300. Accordingly, the information processing device 100 may divide the structure of the molecule of interest 1300 with an appropriate division pattern according to the basis function system, and may improve the accuracy of the quantum chemical calculation. In addition, the information processing device 100 may reduce the processing amount of the quantum chemical calculation.

Next, an example of an overall processing procedure executed by the information processing device 100 will be described with reference to FIG. 15. The overall processing is, for example, an example of the operation of the information processing device 100 depicted in FIGS. 8 to 12. The overall processing is implemented by, for example, the CPU 301, a storage area such as the memory 302 and the recording medium 305, and the network I/F 303 depicted in FIG. 3.

FIG. 15 is a flowchart depicting an example of an overall processing procedure. In FIG. 15, the information processing device 100 obtains the structure of a molecule, the basis function system, and the number of divisions (step S1501). Next, the information processing device 100 generates a graph representing the structure of the obtained molecule (step S1502). Then, based on the obtained basis function system, the information processing device 100 calculates evaluation values representing the magnitude of interactions between atom groups forming the molecule (step S1503).

Next, the information processing device 100 divides the structure of the molecule into multiple fragments by dividing the generated graph, based on the obtained number of divisions and the calculated evaluation values (step S1504). Then, the information processing device 100 generates a molecular structure in which atoms are sorted based on the fragments (step S1505).

Next, the information processing device 100 outputs the structure of the generated molecule and the number of atoms of each fragment (step S1506). Then, the information processing device 100 ends the entire processing. Thus, the information processing device 100 may appropriately divide the structure of the molecule, and may accurately and efficiently perform the quantum chemical calculation related to the molecule using the DMET.

Here, the information processing device 100 may change the sequence of the processes of some steps in the flowchart in FIG. 15. In addition, the information processing device 100 may omit the processes of some steps in the flowchart depicted in FIG. 15.

Next, another example of the operation of the information processing device 100 will be described with reference to FIGS. 16A, 16B, 17A, and 17B. In the examples depicted in FIGS. 8 to 12 described above, a case where the number of divisions is fixed has been described. In the examples depicted in FIGS. 16A, 16B, 17A, and 17B, a case where the information processing device 100 searches for an appropriate number of divisions will be described.

FIGS. 16A, 16B, 17A, and 17B are explanatory diagrams depicting another example of the operation of the information processing device 100. In FIGS. 16A and 16B, similarly to the examples of FIGS. 8 to 12, it is assumed that the information processing device 100 generates a graph representing the structure of a molecule of interest and respectively sets the calculated evaluation values as the weights for the edges included in the graph. In the example depicted in FIGS. 16A and 16B, it is assumed that the molecule of interest is acetanilide. The molecular formula of acetanilide is C₈H₉NO. The basis function system is 6-31G.

Here, the information processing device 100 sets 2 as an initial value of N_{frags}, which indicates the number of divisions. The information processing device 100, based on a result of changing the N_{frags} while dividing the structure of the molecule of interest into the resulting N_{frags} of fragments, repeatedly performs a series of processes including dividing the graph into the resulting N_{frags} of portions until the division fails. Successful division is when there are no empty fragments and there are N_{frags} of fragments each containing at least one atom group. A failure of division is, for example, the presence of an empty fragment.

Specifically, the series of processes includes a process of dividing the graph into the current N_{frags} portions as in the examples depicted in FIGS. 8 to 12. Specifically, the series of processes includes a process of dividing the structure of the molecule of interest into the current N_{frags} of fragments corresponding to the current N_{frags} of portions, as in the examples of FIGS. 8 to 12. Specifically, the series of processes includes a process of adding 1 to N_{frags} when there is no empty fragment as a result of dividing the structure of the molecule of interest into the current N_{frags} of fragments and the division is successful.

When there is an empty fragment as a result of dividing the structure of the molecule of interest into the current N_{frags} of fragments and the division thereby fails, the information processing device 100 does not repeat the series of processes thereafter. As a result, the information processing device 100 may divide the structure of the molecule of interest by each of the multiple N_{frags}. Specifically, the information processing device 100 may divide the structure of the molecule of interest by an appropriate N_{frags}.

The information processing device 100 calculates, for each number of divisions, an index value W_{DMET} representing the degree of contribution to quantum chemical calculation for calculating energy of the molecule of interest using the DMET. The W_{DMET} is evaluated, for example, from the viewpoint of the throughput of the quantum chemical calculation and the viewpoint of the accuracy of the quantum chemical calculation. W_{DMET}=(W_{flag}/max(WL))+(α/(N_{orbs}^{limit}−N_{orbs}^{max})). (W_{flag}/max(WL)) corresponds to a viewpoint of the accuracy of the quantum chemical calculation. (α/(N_{orbs}^{limit}−N_{orbs}^{max})) corresponds to the viewpoint of the processing amount of the quantum chemical calculation. The smaller the value of W_{DMET} is, the larger the degree of contribution is.

Here, W_{flag}=Wₜₒₜₐₗ/N_{frags}. Wₜₒₜₐₗ is the total value of the weights for the cut edges. WL is a list to which W_{flag} is recorded. max(WL) indicates a maximum W_{flag} among the W_{flags} for the respective N_{frags}. α is a coefficient representing the degree of importance of the processing amount of the quantum chemical calculation. N_{orbs}^{limit} is an upper limit value of the number of orbits. N_{orbs}^{max} is the number of orbits in the largest fragment. The largest fragment is, for example, the fragment that contains the largest number of atoms. Graph 1610 in FIG. 16B represents W_{DMET} for each N_{frags}. N_{frags} that minimizes W_{DMET} is 7.

The information processing device 100 determines that N_{frags}=7 that minimizes W_{DMET} is an appropriate number of divisions and selects N_{frags}=7. The information processing device 100 performs quantum chemical calculation for calculating the energy of the molecule of interest using the DMET, based on the selected N_{frags} of fragments obtained by dividing the structure of the molecule of interest. Accordingly, the information processing device 100 may accurately and efficiently perform the quantum chemical calculation for calculating the energy of the molecule of interest using the DMET, with an appropriate number of divisions.

A graph 1600 in FIG. 16A depicts a division error and an execution time in a case where quantum chemical calculation for calculating energy of a molecule of interest using the DMET is performed based on fragments corresponding to N_{frags} obtained by dividing the structure of the molecule of interest. The division error corresponds to the accuracy of the quantum chemical calculation. The division error corresponds to, for example, a bar graph portion of the graph 1600. The execution time corresponds to the processing amount of the quantum chemical calculation. The execution time corresponds to, for example, a line graph portion of the graph 1600.

As depicted in the graphs 1600 and 1610, the information processing device 100 may select N_{frags}=7, which is an appropriate number of divisions that reduces both the division error and the execution time, and may perform the quantum chemical calculation for calculating the energy of the molecule of interest using the DMET. As described, the information processing device 100 may accurately and efficiently perform the quantum chemical calculation for calculating the energy of the molecule of interest using the DMET, with an appropriate number of divisions. Next, FIGS. 17A and 17B will be described.

In FIGS. 17A and 17B, similarly to the examples of FIGS. 8 to 12, it is assumed that the information processing device 100 generates a graph representing the structure of a molecule of interest and respectively sets the calculated evaluation values as the weights for the edges included in the graph. In the examples depicted in FIGS. 17A and 17B, the molecule of interest is tetraethoxysilane. Tetraethoxysilane is also called TEOS (TetraEthyl OrthoSilicate). The basis function system is STO-3G.

Here, similarly to the examples depicted in FIGS. 16A and 16B, the information processing device 100 sets 2 as the initial value of N_{frags} indicating the number of divisions. Similarly to the examples depicted in FIGS. 16A and 16B, the information processing device 100, based on a result of changing the N_{frags} while dividing the structure of the molecule of interest into the resulting N_{frags} of fragments, repeatedly performs a series of processes including dividing the graph into the resulting N_{frags} of portions until the division fails.

As in the example depicted in FIGS. 16A and 16B, when there is an empty fragment as a result of dividing the structure of the molecule of interest into the current N_{frags} of fragments and the division fails, the information processing device 100 does not repeat the series of processes thereafter. As a result, the information processing device 100 may divide the structure of the molecule of interest by each of the multiple N_{frags}. Specifically, the information processing device 100 may divide the structure of the molecule of interest with appropriate N_{frags}.

As in the examples depicted in FIGS. 16A and 16B, the information processing device 100 calculates an index value W_{DMET} representing the degree of contribution to the quantum chemical calculation for calculating the energy of the molecule of interest using the DMET for each number of divisions. A graph 1710 of FIG. 17B represents the W_{DMET} for each N_{frags}. N_{frags} that minimizes W_{DMET} is 4.

The information processing device 100 determines that N_{frags}=4 that minimizes W_{DMET} is an appropriate number of divisions and selects N_{frags}=4. The information processing device 100 performs quantum chemical calculation for calculating the energy of the molecule of interest using the DMET, based on the selected N_{frags} of fragments obtained by dividing the structure of the molecule of interest. Accordingly, the information processing device 100 may accurately and efficiently perform the quantum chemical calculation for calculating the energy of the molecule of interest using the DMET with an appropriate number of divisions.

A graph 1700 in FIG. 17A depicts a division error and an execution time in a case where quantum chemical calculation for calculating energy of the molecule of interest using the DMET is performed based on fragments corresponding to N_{frags} obtained by dividing the structure of the molecule of interest. The division error corresponds to the accuracy of the quantum chemical calculation. The division error corresponds to, for example, a bar graph portion of the graph 1700. The execution time corresponds to the processing amount of the quantum chemical calculation. The execution time corresponds to, for example, a line graph portion of the graph 1700.

As depicted in the graphs 1700 and 1710, the information processing device 100 may select N_{frags}=4, which is an appropriate number of divisions that reduces both the division error and the execution time, and may perform the quantum chemical calculation for calculating the energy of the molecule of interest using the DMET. As described, the information processing device 100 may accurately and efficiently perform the quantum chemical calculation for calculating the energy of the molecule of interest using the DMET, with an appropriate number of divisions.

As depicted in FIGS. 16A, 16B, 17A, and 17B, the information processing device 100 may select a N_{frags} that is an appropriate number of divisions, according to the molecule of interest and the basis function system, and may perform quantum chemical calculation for calculating the energy of the molecule of interest using the DMET. As described, the information processing device 100 may accurately and efficiently perform the quantum chemical calculation for calculating the energy of the molecule of interest using the DMET, with an appropriate number of divisions.

Next, an example of a procedure of a dividing process executed by the information processing device 100 will be described with reference to FIG. 18. The dividing process is, for example, another example of the operation of the information processing device 100 depicted in FIGS. 16A, 16B, 17A, and 17B. The dividing process is implemented by, for example, the CPU 301, a storage area such as the memory 302 and the recording medium 305, and the network I/F 303 depicted in FIG. 3.

FIG. 18 is a flowchart depicting an example of the procedure of the dividing process. In FIG. 18, the information processing device 100 sets N_{frags}=2 (step S1801). Next, the information processing device 100 sets WL={} (step S1802). Then, the information processing device 100 sets OL={} (step S1803). OL is a list in which N_{orbs}^{max} is recorded.

Next, the information processing device 100 divides the structure of the molecule into N_{frags} fragments based on the basis function system (step S1804). Then, the information processing device 100 determines whether there is an empty fragment (step S1805). Here, when there is an empty fragment (step S1805: YES), the information processing device 100 proceeds to the process at step S1809. On the other hand, when there is no empty fragment (step S1805: NO), the information processing device 100 proceeds to the process at step S1806.

At step S1806, the information processing device 100 adds W_{flag}=Wₜₒₜₐₗ/N_{frags} to WL (step S1806). Next, the information processing device 100 adds N_{orbs}^{max} to OL (step S1807). Then, the information processing device 100 increments N_{frags} (step S1808). Thereafter, the information processing device 100 returns to the process at step S1804.

At step S1809, the information processing device 100 calculates W_{DMET}=(W_{flag}/max (WL))+(α/(N_{orbs}^{limit}−N_{orbs}^{max}) for each N_{frags} (step S1809). Next, the information processing device 100 selects N_{frags} that minimizes W_{DMET} (step S1810). Then, the information processing device 100 ends the dividing process. Thus, the information processing device 100 may obtain a result of dividing the structure of the molecule by an appropriate N_{frags}.

Here, the information processing device 100 may change the order of the processes of some steps of the flowchart in FIG. 18. For example, the order of the processes of steps S1801 to S1803 may be interchanged. In addition, the information processing device 100 may omit the processes of some steps of the flowchart in FIG. 18.

The information processing device 100 may be applied to fields such as drug discovery and material development, for example. Specifically, in the field of drug discovery, material development, or the like, the information processing device 100 may be applied to a case where it is desired to perform quantum chemical calculation for calculating the basis energy of a molecule to analyze the structure or properties of the molecule, which is a candidate for a drug or a material. As a result, the information processing device 100 may maintain the accuracy of the quantum chemical calculation while reducing the amount of processing necessary to perform the quantum chemical calculation, may facilitate calculation of the basis energy of the molecule, and may contribute to the fields of drug discovery, material development, and the like.

As described above, according to the information processing device 100, it is possible to obtain a graph that represents the structure of a molecule of interest and includes nodes respectively representing atom groups of multiple atom groups forming the molecule of interest and edges coupling different nodes. The information processing device 100 may calculate, for each edge included in an obtained graph, an evaluation value representing the magnitude of interaction between atom groups represented by different nodes coupled by the edge, based on the atoms belonging to the atom groups. According to the information processing device 100, the calculated evaluation values may be respectively set as the weights for the edges included in the obtained graph. According to the information processing device 100, the structure of the molecule of interest may be divided into multiple fragments corresponding to multiple portions, by dividing the graph into the multiple portions such that the total value of the weights for the edges cut when the graph is divided is minimized. Accordingly, the information processing device 100 may appropriately divide the structure of the molecule of interest into multiple fragments according to the basis function system.

According to the information processing device 100, it is possible to obtain a graph including an edge that couples paired nodes for all pairs of nodes. Accordingly, the information processing device 100 may improve the accuracy of the quantum chemical calculation performed for the molecule of interest using the DMET.

According to the information processing device 100, multiple atom groups may be set so that each atom group includes an atom other than hydrogen and, when there is a hydrogen atom directly coupled to the atom, the hydrogen atom. Thus, the information processing device 100 may reduce the amount of processing for dividing the structure of the molecule of interest into multiple fragments.

According to the information processing device 100, for each edge included in the obtained graph, an evaluation value may be calculated based on the Coulomb interaction distance between atoms belonging to different atom groups among atom groups represented by different nodes coupled by the edge. Accordingly, the information processing device 100 may improve the accuracy of calculating the evaluation value.

According to the information processing device 100, for each edge included in the obtained graph, an evaluation value may be calculated based on the bath orbit eigenvalue in a case where atom groups represented by different nodes coupled by the edge are fragments. Accordingly, the information processing device 100 may improve the accuracy of calculating the evaluation value.

According to the information processing device 100, the graph may be divided into portions corresponding to the number of candidates for each of the multiple numbers of candidates for dividing the structure of the molecule of interest. According to the information processing device 100, for each number of candidates, the structure of the molecule of interest may be divided into fragments corresponding to the number of candidates obtained by dividing the graph. According to the information processing device 100, it is possible to output fragments corresponding to any candidate number determined to have the highest contribution to the quantum chemical calculation using the DMET, among the multiple numbers of candidates (multiple candidate numbers). Accordingly, the information processing device 100 may divide the graph by an appropriate number of candidates, among the multiple numbers of candidates. The information processing device 100 may divide the structure of the molecule of interest into an appropriate number of candidates.

According to the information processing device 100, it is possible to receive designation of the number of divisions of the structure of the molecule of interest. According to the information processing device 100, the structure of the molecule of interest may be divided into the number of fragments corresponding to the number of designated portions by dividing the graph into the number of designated portions. Thus, the information processing device 100 may divide the structure of the molecule of interest into a number of fragments desired by the user.

According to the information processing device 100, it is possible to output information indicating each atom of one or more atoms belonging to a fragment in association with information indicating each fragment of multiple fragments. Accordingly, the information processing device 100 may make information indicating each fragment and information indicating each atom of one or more atoms belonging to the fragment available to the outside. The information processing device 100 may make it easy to externally perform quantum chemical calculation for a molecule of interest using the DMET.

According to the information processing device 100, it is possible to perform quantum chemical calculation using the DMET on the basis of multiple fragments. Accordingly, the information processing device 100 may obtain a result of performing the quantum chemical calculation using the DMET.

According to the information processing device 100, multiple atom groups may be set so that each atom group includes only one atom. Thus, the information processing device 100 may improve the accuracy of dividing the structure of the molecule of interest into multiple fragments. The information processing device 100 may reduce the amount of processing necessary to distribute multiple atoms to multiple atom groups.

The information processing method described in the present embodiment may be implemented by executing a prepared program on a computer such as a personal computer and a workstation. The program is stored on a non-transitory, computer-readable recording medium such as a hard disk, a flexible disk, a compact disc read-only memory (CD-ROM), a magneto-optical (MO) disc, and a digital versatile disc (DVD), read out from the computer-readable medium, and executed by the computer. The program may be distributed through a network such as the Internet.

According to one aspect of the present disclosure, it is possible to improve the accuracy of calculating molecular energies using the density matrix embedding theory.

All examples and conditional language provided herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A computer-readable recording medium storing therein a program for causing a computer to execute a process, the process comprising:
obtaining a graph representing a structure of a molecule of interest, the graph including a plurality of nodes respectively representing a plurality of atom groups of a plurality of atoms forming the molecule of interest, the graph further including a plurality of edges each coupling different nodes of the plurality of nodes;
calculating a plurality of evaluation values respectively for the plurality of edges included in the obtained graph, each of the plurality of evaluation values representing a magnitude of an interaction between atom groups represented by the different nodes coupled by the each of the plurality of edges, the each of the plurality of evaluation values being calculated based on atoms belonging to the atom groups represented by the different nodes coupled by the each of the plurality of edges; and
dividing the graph into a plurality of portions when the calculated plurality of evaluation values is set as a plurality of weights respectively for each of the plurality of edges included in the obtained graph, the graph being divided into the plurality of portions by cutting any of the plurality of edges such that a sum of respective weights thereof is minimized thereby dividing the structure of the molecule of interest into a plurality of fragments corresponding to the plurality of portions.

2. The computer-readable recording medium according to claim 1, wherein the obtaining includes obtaining the graph including the plurality of edges coupling all pairs of the plurality of nodes.

3. The computer-readable recording medium according to claim 2, wherein each of the plurality of atom groups includes an atom other than hydrogen and when a hydrogen atom is directly coupled to the atom other than hydrogen, the hydrogen atom.

4. The computer-readable recording medium according to claim 3, wherein the calculating includes calculating the evaluation value for each of the plurality of edges included in the obtained graph, based on a Coulomb interaction distance between the atoms belonging to the atom groups represented by the different nodes coupled by the each of the plurality of edges.

5. The computer-readable recording medium according to claim 3, wherein the calculating includes:
calculating the evaluation value for each of the plurality of edges included in the obtained graph, based on a bath orbit eigenvalue when the atom groups represented by the different nodes coupled by the edge are fragments.

6. The computer-readable recording medium according to claim 1, wherein
the dividing includes dividing the graph into the plurality of portions corresponding to each of a plurality of candidate numbers for dividing the structure of the molecule of interest, thereby dividing the structure of the molecule of interest into the plurality of fragments corresponding in number to the each of the plurality of candidate numbers, and
the process further comprises outputting the plurality of fragments corresponding in number to a candidate number that of the plurality of candidate numbers is determined to have a highest contribution to a quantum chemical calculation using a density matrix embedding theory, the plurality of fragments being obtained by dividing the structure of the molecule of interest.

7. The computer-readable recording medium according to claim 1, the process further comprising receiving designation of a number of divisions of the structure of the molecule of interest, wherein
the dividing includes dividing the graph into the plurality of portions of the number for which the designation is received thereby dividing the structure of the molecule of interest into the plurality of fragments corresponding in number to the plurality of portions.

8. The computer-readable recording medium according to claim 1, the process further comprising associating and outputting information indicating each of the plurality of fragments with information indicating each of one or more atoms belonging to the each of the plurality of fragments.

9. The computer-readable recording medium according to claim 1, the process further comprising performing a quantum chemical calculation using a density matrix embedding theory based on the plurality of fragments.

10. The computer-readable storage medium according to claim 1, wherein each of the atom groups includes only one atom.

11. An information processing method executed by a computer, the method comprising:
obtaining a graph representing a structure of a molecule of interest, the graph including a plurality of nodes respectively representing a plurality of atom groups of a plurality of atoms forming the molecule of interest, the graph further including a plurality of edges each coupling different nodes of the plurality of nodes;
calculating a plurality of evaluation values respectively for the plurality of edges included in the obtained graph, each of the plurality of evaluation values representing a magnitude of an interaction between atom groups represented by the different nodes coupled by the each of the plurality of edges, the each of the plurality of evaluation values being calculated based on atoms belonging to the atom groups represented by the different nodes coupled by the each of the plurality of edges; and
dividing the graph into a plurality of portions when the calculated plurality of evaluation values is set as a plurality of weights respectively for each of the plurality of edges included in the obtained graph, the graph being divided into the plurality of portions by cutting any of the plurality of edges such that a sum of respective weights thereof is minimized thereby dividing the structure of the molecule of interest into a plurality of fragments corresponding to the plurality of portions.

12. An information processing device, comprising:
a memory; and
a processor coupled to the memory, the processor configured to:
obtain a graph representing a structure of a molecule of interest, the graph including a plurality of nodes respectively representing a plurality of atom groups of a plurality of atoms forming the molecule of interest, the graph further including a plurality of edges each coupling different nodes of the plurality of nodes;
calculate a plurality of evaluation values respectively for the plurality of edges included in the obtained graph, each of the plurality of evaluation values representing a magnitude of an interaction between atom groups represented by the different nodes coupled by the each of the plurality of edges, the each of the plurality of evaluation values being calculated based on atoms belonging to the atom groups represented by the different nodes coupled by the each of the plurality of edges; and
divide the graph into a plurality of portions when the calculated plurality of evaluation values is set as a plurality of weights respectively for each of the plurality of edges included in the obtained graph, the graph being divided into the plurality of portions by cutting any of the plurality of edges such that a sum of respective weights thereof is minimized thereby dividing the structure of the molecule of interest into a plurality of fragments corresponding to the plurality of portions.
